(19) United States
(12) Patent Application Publication
(10) Pub. No.: US 2018/0161279 A1
(43) Pub. Date: Jun. 14, 2018

(54) GASTRO-RETENTIVE MODIFIED RELEASE DOSAGE FORMS FOR OPROZOMIB AND PROCESS TO MAKE THEREOF

(71) Applicant: AMGEN INC., Thousand Oaks, CA (US)

(72) Inventors: John Inn CHUNG, Porter Ranch, CA (US); Armen PIRJANIAN, Sunland, CA (US); Fernando Antonio ALVAREZ-NUNEZ, Newbury Park, CA (US); Jeffrey Michael KATZ, Oak Park, CA (US); Dominick Paul DAURIO, Camarillo, CA (US); Stevedat LA, Moorpark, CA (US); Michael T. KENNEDY, Newbury Park, CA (US)

(21) Appl. No.: 15/840,908

(22) Filed: Dec. 13, 2017

Related U.S. Application Data
(60) Provisional application No. 62/434,250, filed on Dec. 14, 2016.

(51) Int. Cl. A61K 9/20; A61K 9/00; A61K 9/28; A61K 38/06

(52) U.S. Cl. CPC A61K 9/2031; A61K 9/2086; A61K 9/0065; A61K 38/06; A61K 9/2013; A61K 9/28; A61K 9/2054

(57) ABSTRACT

This disclosure features gastro-retentive (GR) modified release pharmaceutical dosage forms (e.g., solid dosage forms, e.g., tablets, e.g., bilayer tablets) that are useful for the oral administration of oprozomib, or a pharmaceutically acceptable salt thereof, to a human or animal subject as well as methods of making and using the dosage form.

### RELATED APPLICATIONS

This application claims the benefit of U.S. Provisional patent application 62/434,250 filed on Dec. 14, 2016, which specification is hereby incorporated herein by reference in its entirety for all purposes.

### TECHNICAL FIELD

This disclosure features pharmaceutical dosage forms (e.g., gastro-retentive (“GR”) modified release pharmaceutical dosage forms; e.g., solid dosage forms, e.g., tablets) that are useful for the oral administration of oprozomib, or a pharmaceutically acceptable salt thereof, to a human or animal subject as well as methods of making and using the dosage forms. A bilayer tablet dosage form and process was developed that produces an gastro-retentive modified release tablet that erodes slowly, thus allowing for absorption of the sparingly soluble active pharmaceutical agent in the upper regions of the small intestine, such as the duodenum and jejunum region, of the gastrointestinal tract, thereby decreasing the adverse effects of nausea, vomiting and/or diarrhea.

### BACKGROUND

The proteasome has been validated as a therapeutic target, as demonstrated by the FDA approval of bortezomib, a boronic acid proteasome inhibitor, for the treatment of various cancer indications, including multiple myeloma; and more recently, carfilzomib, a tetra-peptide epoxy ketone-containing proteasome inhibitor, for the treatment of refractory multiple myeloma.

Oprozomib (chemical structure shown below) is an orally bioavailable (epoxy ketone-containing) tri-peptide irreversible proteasome inhibitor, which has demonstrated preclinical anti-tumor activity and a broad therapeutic window in preclinical models and is currently being studied in Phase I clinical trials.

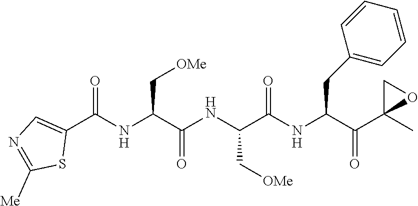 [SMILES: COC[C@H](NC(=O)c1cnc(C)s1)C(=O)N[C@@H](COC)C(=O)N[C@@H](Cc1ccccc1)C(=O)[C@@]1(C)CO1]

### Oprozomib

This invention is an improvement over the modified-release oral drug dosage forms described in U.S. Pat. No. 5,007,790 and U.S. Pat. No. 5,582,837. The dosage forms described therein consist of a plurality of solid particles composed of a solid drug dispersed in either a crosslinked or non-crosslinked, hydrophilic, water-swellable polymer. In these dosage forms, the polymers in which the drug is dispersed imbibe water, causing the particles to swell which promotes their retention and erode allowing the drug to be released and then dissolved. Poly(ethylene oxide) and hydroxypropyl methylcellulose polymers have been used in the pharmaceutical industry for controlled drug delivery systems including, for example, gastric retentive, oral drug delivery systems (see U.S. Pat. No. 5,972,389).

However, compared to known practices, the present invention GR modified release dosage form utilizes one or more different grades, preferably two, of polyethylene oxide to develop a bilayer GR modified release tablet. The prior art does not disclose the use of these polymers with a tri-peptide irreversible proteasome inhibitor, such as oprozomib, in a bilayer tablet wherein the active pharmaceutical agent, such as oprozomib, is contained within only one layer.

### SUMMARY

The present disclosure features pharmaceutical dosage forms (e.g., gastro-retentive modified release pharmaceutical dosage forms; e.g., solid dosage forms, e.g., tablets, e.g., bilayer tablets) that are useful for the oral administration of oprozomib, or a pharmaceutically acceptable salt thereof, to a human or animal subject as well as methods of making and using the dosage forms.

In contrast to the systems of the prior art, the present invention provides erodible, gastric-retentive modified drug dosage forms for the delivery of sparingly soluble drugs, such as a peptide, preferably a tri-peptide irreversible proteasome inhibitor, more preferably oprozomib. More particularly, the present invention provides swellable polymers designed to deliver sparingly soluble drugs into the gastrointestinal (“G.I.” or “GI”) tract as a result of the gradual erosion of the polymer. The dosage forms described herein consist of two layers (bilayer). One layer consists of a drug and polymer to control the release of the drug and a second layer which consists of a polymer to help the dosage remain in the stomach for an extended period of time with the coadminstration of food. In the drug layer, the drug is dispersed within the polymer. As the polymer erodes, drug is released and can dissolve.

Moreover, the swelling properties of the polymers are important in that they allow the dosage forms to be retained in the stomach where they effectively deliver drugs on a continuous basis to the stomach, duodenum, jejunum, and upper sections of the small intestine where absorption is efficient. The beneficial properties of the swelling polymers include protecting drugs against the degradative environment of the G.I. tract, overcoming a too rapid drug release rate, or targeting the absorption of drugs to specific areas within the G.I. tract.

In one embodiment of the present invention, the present invention comprises a gastro-retentive modified release oral drug dosage form for releasing a sparingly soluble active pharmaceutical agent into the stomach, duodenum and/or upper small intestine of a patient, the drug dosage form comprising:
a. a first layer, the first layer comprising a first swellable polymer;
b. a second layer, the second layer comprising a second swellable polymer;
c. the first swellable polymer swells via imbibition of water from gastric fluid to promote gastric retention in the stomach of the patient;
d. the second swellable polymer swells via imbibition of water from gastric fluid to promote gastric retention in the stomach of the patient;
e. each first and second swellable polymer gradually erodes over a time period of hours, the erosion commencing upon contact with the gastric fluid, wherein the erosion releases the sparingly soluble pharmaceutical agent to the stomach, duodenum and/or upper small intestine of the patient as a result of the erosion at a rate corresponding to the time period;
f. wherein the first and second swellable polymers each comprises polyethylene oxide;
g. a sparingly soluble active pharmaceutical agent dispersed within at least one of the first and second layers; and
h. wherein the sparingly soluble active pharmaceutical agent is oprozomib or a pharmaceutically acceptable salt thereof.

In another embodiment, the first swellable polymer decreases the release rate of the sparingly soluble active pharmaceutical agent.

In another embodiment, the first layer is granulated.

In another embodiment, the oprozomib is dispersed within the first layer.

In another embodiment, the oprozomib is dispersed within the first swellable polymer.

In another embodiment, the first and second swellable polymers exhibit different swelling and erosion rates.

In another embodiment, the second swellable polymer swells at a faster rate and erodes at a slower rate than the first polymer.

In another embodiment, the dosage form is in a form suitable for oral administration.

In another embodiment, the dosage form is a solid tablet.

In another embodiment, the dosage form is a bilayer tablet.

In another embodiment, the patient is in a fed mode.

In another embodiment, the dosage form comprises from about 2 weight percent to about 50 weight percent of oprozomib, or a pharmaceutically acceptable salt thereof.

In another embodiment, the dosage form comprises from about 3 weight percent to about 30 weight percent of oprozomib, or a pharmaceutically acceptable salt thereof.

In another embodiment, the dosage form comprises from about 4 weight percent to about 10 weight percent of oprozomib, or a pharmaceutically acceptable salt thereof.

In another embodiment, the dosage form comprises about 4.17 weight percent of oprozomib, or a pharmaceutically acceptable salt thereof.

In another embodiment, the dosage form comprises about 25 milligrams of oprozomib, or a pharmaceutically acceptable salt thereof.

In another embodiment, the dosage form comprises from about 4.17 weight percent to about 5 weight percent of oprozomib, or a pharmaceutically acceptable salt thereof and about 25 milligrams of oprozomib, or a pharmaceutically acceptable salt thereof.

In another embodiment, the dosage form comprises about 4.17 weight percent of oprozomib, or a pharmaceutically acceptable salt thereof and about 25 milligrams of oprozomib, or a pharmaceutically acceptable salt thereof.

In another embodiment, the dosage form comprises about 16.67 weight percent of oprozomib, or a pharmaceutically acceptable salt thereof.

In another embodiment, the dosage form comprises from about 20 weight percent to about 25 weight percent of oprozomib, or a pharmaceutically acceptable salt thereof.

In another embodiment, the dosage form comprises about 20 weight percent of oprozomib, or a pharmaceutically acceptable salt thereof.

In another embodiment, wherein the dosage form comprises about 150 milligrams of oprozomib, or a pharmaceutically acceptable salt thereof.

In another embodiment, the dosage form comprises from about 20 weight percent to about 25 weight percent of oprozomib, or a pharmaceutically acceptable salt thereof; and about 150 milligrams of oprozomib, or a pharmaceutically acceptable salt thereof.

In another embodiment, the dosage form comprises about 25 weight percent of oprozomib, or a pharmaceutically acceptable salt thereof; and about 200 milligrams of oprozomib, or a pharmaceutically acceptable salt thereof.

In another embodiment, the dosage form comprises about 33.33 weight percent of oprozomib, or a pharmaceutically acceptable salt thereof.

In another embodiment, the dosage form comprises about 100 milligrams of oprozomib, or a pharmaceutically acceptable salt thereof.

In another embodiment, the dosage form comprises about 300 milligrams of oprozomib, or a pharmaceutically acceptable salt thereof.

In another embodiment, the dosage form comprises about 600 milligrams of oprozomib, or a pharmaceutically acceptable salt thereof.

In another embodiment, the dosage form comprises about 50 weight percent of oprozomib, or a pharmaceutically acceptable salt thereof; and about 300 milligrams of oprozomib, or a pharmaceutically acceptable salt thereof.

In another embodiment, the dosage form comprises about 50 weight percent of oprozomib, or a pharmaceutically acceptable salt thereof; and about 600 milligrams of oprozomib, or a pharmaceutically acceptable salt thereof.

In another embodiment, the dosage form comprises from about 15 weight percent to about 20 weight percent of oprozomib, or a pharmaceutically acceptable salt thereof; and about 100 milligrams of oprozomib, or a pharmaceutically acceptable salt thereof.

In another embodiment, the dosage form comprises about 16.67 weight percent of oprozomib, or a pharmaceutically acceptable salt thereof; and about 100 milligrams of oprozomib, or a pharmaceutically acceptable salt thereof.

In another embodiment, the dosage form comprises about 20 weight percent of oprozomib, or a pharmaceutically acceptable salt thereof; and about 150 milligrams of oprozomib, or a pharmaceutically acceptable salt thereof.

In another embodiment, wherein the dosage form comprises about 24.24 weight percent of oprozomib, or a pharmaceutically acceptable salt thereof; and about 200 milligrams of oprozomib, or a pharmaceutically acceptable salt thereof.

In another embodiment, the oprozomib, or pharmaceutically acceptable salt thereof, is a crystalline solid.

In another embodiment, the oprozomib, or pharmaceutically acceptable salt thereof, is an amorphous solid.

In another embodiment, the dosage form further comprises one or more fillers.

In another embodiment, the one or more fillers is microcrystalline cellulose.

In another embodiment, the first swellable polymer comprises from about 2 weight percent to about 80 weight percent of the total weight of the dosage form.

In another embodiment, the first swellable polymer comprises from about 15 weight percent to about 75 weight percent of the total weight of the dosage form.

In another embodiment, the first swellable polymer comprises from about 5 weight percent to about 20 weight percent of the total weight of the dosage form.

In another embodiment, the polyethylene oxide is PolyOx WSR 1105 LEO® polymer.

In another embodiment, the second swellable polymer comprises from about 2 weight percent to about 80 weight percent of the total weight of the dosage form.

In another embodiment, the second swellable polymer comprises from about 10 weight percent to about 65 weight percent of the total weight of the dosage form. In another embodiment, the second swellable polymer comprises from about 16 weight percent to about 30 weight percent of the total weight of the dosage form.

In another embodiment, the second swellable polymer has a greater molecular weight than the first swellable polymer.

In another embodiment, the dosage form further comprises one or more lubricants.

In another embodiment, the one or more lubricants is magnesium stearate.

In another embodiment, the dosage form further comprises one or more coatings.

In another embodiment, the one or more coatings is Opadry II White® (85F18422) coating.

In another embodiment, the tablet has a thickness of from about 2.5 millimeters to about 8 millimeters.

In another embodiment, the tablet has a thickness of from about 2.5 millimeters to about 4 millimeters.

In another embodiment, the tablet has a hardness of from about 1.00 kp to about 50.00 kp.

In another embodiment, the dosage form comprises:
a. a first layer comprising, by total weight percent (wt %) of the dosage form,
   i) about 4% oprozomib;
   ii) about 62% microcrystalline cellulose;
   iii) about 12% PolyOx® WSR 1105 LEO® polymer; and
   iv) about 0.50% magnesium stearate;
b. a second layer comprising, by total weight percent (wt %) of the dosage form,
   i) about 21% PolyOx® WSR 303 LEO® polymer; and
   ii) about 0.50% magnesium stearate; and
c. a film coating.

In another embodiment, the dosage form comprises;
a. a first layer, comprising, by total weight percent (wt %) of the dosage form:
   i) about 17% oprozomib;
   ii) about 49% microcrystalline cellulose;
   iii) about 8% PolyOx® WSR 1105 LEO® polymer; and
   iv) about 0.50% magnesium stearate;
b. a second layer, comprising, by total weight percent (wt %) of the dosage form:
   i) about 25% PolyOx® WSR 303 LOE® polymer; and
   ii) about 0.50% magnesium stearate; and
c. a film coating.

In another embodiment, the dosage form comprises;
a. a first layer, comprising, by total weight percent (wt %) of the dosage form:
   i) about 20% oprozomib;
   ii) about 49% microcrystalline cellulose;
   iii) about 11% PolyOx® WSR 1105 LEO® polymer; and
   iv) about 0.8% magnesium stearate;
b. a second layer, comprising, by total weight percent (wt %) of the dosage form:
   i) about 20% PolyOx® WSR 303 LOE® polymer; and
   ii) about 0.2% magnesium stearate; and
c. a film coating.

In another embodiment, the dosage form comprises;
a. a first layer, comprising, by total weight percent (wt %) of the dosage form:
   i) about 24% oprozomib;
   ii) about 48% microcrystalline cellulose;
   iii) about 9% PolyOx® WSR 1105 LEO® polymer; and
   iv) about 0.8% magnesium stearate;
b. a second layer, comprising, by total weight percent (wt %) of the dosage form:
   i) about 18% PolyOx® WSR 303 LOE® polymer; and
   ii) about 0.2% magnesium stearate; and
c. a film coating.

In another embodiment, the dosage form provides oprozomib with time to peak plasma concentrations of from about 1 hour to about 8 hours.

In another embodiment, the dosage form provides oprozomib with time to peak plasma concentrations of from about 4 hours to about 8 hours.

In another embodiment, the dosage form provides oprozomib with time to peak plasma concentrations of from about 4 hours to about 6 hours.

In another embodiment, the dosage form provides oprozomib with time to peak plasma concentrations of about 8 hours.

In another embodiment, the dosage form provides oprozomib from about less than or equal to 40% of a preferred dose at approximately 1 hour.

In another embodiment, the dosage form provides oprozomib from about 40% to about 75% of the preferred dose at approximately 4 hours.

In another embodiment, the dosage form provides oprozomib from about greater than or equal to 75% of the preferred dose at approximately 8 hours.

In another embodiment, a single dose of the dosage form comprising about 60 mg of oprozomib to a dog produces peak plasma concentration (Cₘₐₓ) of oprozomib of 3.81 ng/mL (having a standard deviation of 2.28).

In another embodiment, a single dose of the dosage form to a dog produces an area under the concentration time curve to the last time point (AUC) of oprozomib of 15.6 ng*hr/mL (having a standard deviation of 17.4).

In another embodiment, the dosage form is stable upon actual or simulated storage at 25° C./60% relative humidity for at least 1 month.

In another embodiment, equal to or more than about 75% of oprozomib, or a pharmaceutically acceptable salt thereof, is released within about 8 hours as determined by UV under the following dissolution conditions:

| | |
|---|---|
| Dissolution medium | About 0.01N Hydrochloric acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all rows |
| Residence Time | 2 hrs in first row |
| | 3 hrs in second row |
| | 3 hrs in third row |
| Sampling | Manual or automatic |
| Sampling Time | About 1, 2, 3, 4, 5, 7, 8 hours |
| Sampling Volume | About 5 mL |
| Screen size | 20 mesh |
| Filter | 5 um Titan 2 syringe filters (used new |
| | filters for each sampling time point) |
| UV Detection | 258 nm |
| Cell path length | 2 mm for 100 mg |
| (for standard UV | 10 mm for 25 mg |
| spectrophotometer) | |

In another embodiment, equal to or more than about 75% of oprozomib, or a pharmaceutically acceptable salt thereof, is released within about 8 hours as determined by UV under the following dissolution conditions:

| | |
|---|---|
| Dissolution medium | About 0.01N Hydrochloric |
| | acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all |
| | rows |
| Residence Time | 2 hrs in first row |
| | 2 hrs in second row |
| | 2 hrs in third row |
| | 2 hrs in fourth row |
| Sampling | Manual |
| Sampling Time | About 1, 2, 3, 4, 5, 7, 8 hours |
| Sampling Volume | About 5 mL |
| Screen size | 20 mesh |
| Filter | 5 um Titan 2 syringe filters |
| | (used new filters for each sampling |
| | time point) |
| UV Detection | 258 nm |
| Cell path length (for | 1 mm for 150 mg and 200 mg |
| standard UV | 2 mm for 100 mg |
| spectrophotometer Cary ® UV | 10 mm for 25 mg |
| 50, Varian, Inc.)) | |

In another embodiment, from about less than or equal to 40% of a preferred dose of oprozomib is released at approximately 1 hour.

In another embodiment, from about 40% to about 75% of the preferred dose of oprozomib is released at approximately 4 hours.

In another embodiment, the dosage form is prepared by dry granulation.

Particulate, insoluble matter suitable for use in this embodiment also includes solid particles that are granulations of a selected drug with an agent that serves to delay dissolution of the granules until they have passed out of the acidic environment of the stomach. Such “enteric coated” agents include, but are not limited to, methacrylic acid copolymer, types A, B, or C, which are commercially available from Rohm Tech, Inc. (Malden, Mass.), and water-based dispersions of cellulose acetate phthalate latex, which is commercially available from Eastman Fine Chemicals (Kingsport, Tenn.). As such, in yet another embodiment, the present invention provides a gastro-retentive modified release oral drug dosage form for releasing an enteric-coated drug into the stomach of a patient.

Nausea, vomiting and diarrhea (“NVD”) is a gastrointestinal side effect that has been observed with oral administration of oprozomib, e.g., when oprozomib is formulated as a solution, a suspension, and in capsule and gastro-retentive release tablet forms. In-vivo animal studies have suggested that the NVD effect of oprozomib was the result of local proteasome inhibition, and that this local proteasome inhibition was not site (stomach or small intestine) specific. Rather, the NVD effect of oprozomib appeared to depend on the local concentration of oprozomib in the gastrointestinal (GI) tract (e.g., the stomach, duodenum, jejunum, ileum, and colon). Once oprozomib enters the lower GI tract, it is not absorbed well thus allowing oprozomib to cause GI tolerability issues. This implied that the occurrence of high local oprozomib concentrations in the stomach, duodenum and/or jejunum regions of the GI tract would likely trigger some degree of NVD in patients and potentially impact dose escalation.

In another embodiment, wherein the dosage form provides a reduced incidence or severity of one or more gastrointestinal (GI) side effects.

In another embodiment, one or more gastrointestinal (GI) side effects include one or more of nausea, vomiting or emesis, increased salivation and diarrhea.

In another embodiment, the side effects include vomiting or emesis.

In another embodiment, the side effects include increased salivation.

In another embodiment, the present invention discloses a method for treating a disease or condition selected from the group consisting of cancer, autoimmune disease, graft or transplant-related condition, neurodegenerative disease, fibrotic-associated condition, ischemic-related conditions, infection (viral, parasitic or prokaryotic) and diseases associated with bone loss, the method comprising administering a dosage form as disclosed herein.

In another embodiment, the disease or condition is cancer.

In another embodiment, the cancer is selected from multiple myeloma, Waldenström's macroglobulinemia, chronic lymphocytic leukemia, and myelodysplastic syndromes.

In another embodiment, the present invention comprises two different dosage strengths (25 mg and 100 mg), as described in Table 1.

In another embodiment, the present invention comprises two different dosage strengths (150 mg and 200 mg), as described in Table 1a.

The details of one or more embodiments of the invention are set forth in the accompanying drawings and the description below. Other features and advantages of the formulations and methods of making and using the same will be apparent from the description and drawings, and from the claims.

### DESCRIPTION OF DRAWINGS

FIG. 1 is a graph showing the in vitro release profile of the present GR modified release dosage form (25 mg, Table 1) compared to the immediate release (“IR”) dosage form (FIG. 11).

FIG. 2 shows an XRPD (X-ray powder diffraction) pattern of a crystalline form of oprozomib that is described in, e.g., U.S. Pat. No. 9,295,708.

FIG. 3 shows a DSC (differential scanning calorimetry) thermogram of a crystalline form of oprozomib that is described in, e.g., U.S. Pat. No. 9,295,708.

FIG. 4 shows a thermogravimetric (TG) thermogram of a crystalline form of oprozomib described in U.S. Pat. No. 9,295,708.

FIG. 5 shows the experimental design for the pharmacokinetic and pharmacodynamics (PK/PD) studies for the administration of the oprozomib dosage forms to dogs.

FIG. 6 shows pharmacokinetic (PK) data obtained for IR (FIG. 11) and GR2 (FIG. 13) oprozomib dosage forms when administered to dogs.

FIG. 7 shows pharmacodynamic (PD) data obtained for IR (FIG. 11) and GR2 (FIG. 13) oprozomib dosage forms when administered to dogs.

FIG. 8 shows pharmacokinetic (PK) data obtained for GR1 tablet (FIG. 12), GR2 tablet (FIG. 13), mini-tablet (FIG. 14), and IR tablet (FIG. 11) oprozomib dosage forms when administered to dogs on Day 1.

FIG. 9 shows pharmacokinetic (PK) data obtained GR1 (FIG. 12) tablet, GR2 tablet (FIG. 13), mini-tablet (FIG. 14) and IR tablet (FIG. 11) oprozomib dosage forms when administered to dogs on Day 8.

FIG. 10 shows proteasome activity data obtained for IR (FIG. 11), GR1 (FIG. 12), GR2 (FIG. 13) and mini-tablet (FIG. 14) oprozomib dosage forms when administered to dogs.

FIG. 10(a) shows proteasome activity data obtained for IR (FIG. 11), GR1 (FIG. 12), GR2 (FIG. 13) and mini-tablet (FIG. 14) oprozomib dosage forms when administered to dogs.

FIG. 11 shows the composition of the immediate release dosage form (IR).

FIG. 12 shows the composition of the gastro-retentive monolithic dosage form (GR1).

FIG. 13 shows the composition of the gastro-retentive bilayer tablet dosage form (GR2).

FIG. 14 illustrates the composition of the mini-tablet dosage form.

FIG. 15 shows emesis events following oral administration of different oprozomib dosage forms.

FIG. 16 shows the manufacturing flow diagram for the GR modified release bilayer tablets.

FIG. 17 illustrates the Dissolution Profile for GR modified release Development Tablets 25-mg oprozomib dose.

FIG. 18 illustrates the Dissolution Profile for GR modified release Development Tablets 100-mg oprozomib dose.

FIG. 19 illustrates the Dissolution Profile for GR modified release Clinical Tablets 25-mg oprozomib dose.

FIG. 20 illustrates the Dissolution Profile for GR modified release Clinical Tablets 100-mg oprozomib dose.

FIG. 21 shows the composition of the gastro-retentive bilayer tablet dosage form (GR3).

FIG. 22 shows the composition of the gastro-retentive bilayer tablet dosage form (GR4).

FIG. 23 illustrates the Dissolution Profile for GR3 modified release Clinical Tablets 100-mg oprozomib dose (600 mg).

FIG. 24 illustrates the Dissolution Profile for GR4 modified release Clinical Tablets 150-mg oprozomib dose (825 mg).

### DETAILED DESCRIPTION

The following abbreviations may be used herein:
˜ about
Calcd Calculated
ee or e.e. enantiomeric excess
eq Equivalent
Et Ethyl
g gram(s)
h, or hrs hour(s)
H₂O Water
HCl hydrochloric acid
HPLC high performance liquid chromatography
L liter(s)
LCMS LC-MS or LC/MS liquid chromatography mass spectrometry
M molar (mol L⁻¹)
Me methyl
mg milligram(s)
min minute(s)
mL milliliter(s)
MS mass spectrometry
m/z mass-to-charge ratio
N Normality (Eq/L)
N₂ nitrogen gas
NaCl sodium chloride
NMR nuclear magnetic resonance spectroscopy
ppm parts per million
QD once daily
QNMR quantitative NMR
RT or rt or r.t. room temperature
sat. or sat'd or satd Saturated
SiO₂ silicon dioxide, silica
THF Tetrahydrofuran
θ Theta
TLC thin layer chromatography
v/v volume per volume
w/w weight per weight

It is noted that when a percent (%) is used with regard to a liquid, it is a percent by volume with respect to the solution. When used with a solid, it is the percent with regard to the solid composition.

The symbol “—” represents a covalent bond and can also be used in a radical group to indicate the point of attachment to another group. In chemical structures, the symbol — is commonly used to represent a methyl group in a molecule.

As used herein, chemical structures which contain one or more stereocenters depicted with dashed and bold bonds (i.e., [symbol] and [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures that include one or more stereocenters which are illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

The term “compound”, as used herein is meant to include all stereoisomers, geometric isomers, tautomers, and isotopes of the structures depicted. Compounds herein identified by name or structure as one particular tautomeric form are intended to include other tautomeric forms unless otherwise specified.

All compounds, and pharmaceutically acceptable salts thereof, can be found together with other substances such as water and solvents (e.g., hydrates and solvates).

The term “excipient”, as used herein, means any pharmaceutically acceptable additive, carrier, diluent, adjuvant or other ingredient, other than the active pharmaceutical ingredient (API), which is typically included for formulation and/or administration to a patient. Handbook of Pharmaceutical Excipients, 5^{th} Edition, R. C. Rowe, P. J. Sheskey, and S. C. Owen, editors, Pharmaceutical Press, 2005, Hardback, 928, 0853696187.

For the terms “for example” and “such as” and grammatical equivalences thereof, the phrase “and without limitation” is understood to follow unless explicitly stated otherwise.

The term “modified release”, as used herein, means the dosage forms may be formulated such that the drug release is modified.

There are two types of modified-release products: delayed-release, and extended-release.

The term “delayed-release”, as used herein, means the dosage forms may be are formulated with acid-resistant or enteric coatings to protect acid-labile drug substances from the gastric environment or to prevent adverse events such as irritation. Delayed release of the drug substance may also occur by means of formulation such as gastroretentive technology.

The term “extended-release”, as used herein, means the dosage forms may be are formulated in such a manner as to make the drug substance available over an extended period of time following ingestion.

The term “patient” means subjects including animals, such as dogs, cats, cows, horses, sheep and humans. Particular patients are mammals. The term patient includes males and females.

The term “patient in need” means a patient having, or at risk of having, one or more diseases or conditions is involved, such as cancers. Identifying a patient in need can be in the judgment of a subject or a health care professional and can be subjective (e.g., opinion) or objective (e.g., measurable by a test or diagnostic method).

The term “pharmaceutically acceptable” is employed herein to refer to those ligands, materials, compositions, and/or dosage forms, which are, within the scope of sound medical judgment, suitable for administration to a patient, commensurate with a reasonable benefit/risk ratio.

The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

The phrases “systemic administration”, “administered systemically”, “peripheral administration”, and “administered peripherally” as used herein mean the administration of a ligand, drug, or other material via route other than directly into the central nervous system, such that it enters the patient's system and thus, is subject to metabolism and other like processes, for example, subcutaneous administration.

The term “therapeutically effective amount” means an amount of a compound that ameliorates, attenuates or eliminates one or more symptom of a particular disease or condition, or prevents or delays the onset of one of more symptom of a particular disease or condition.

The terms “treating”, “treat” or “treatment” and the like include preventative (e.g., prophylactic) and palliative treatment.

The methods provided herein include the manufacture and use of pharmaceutical dosage forms, which include one or more of the compounds provided herein. Also included are the pharmaceutical dosage forms themselves.

The dosage form components, such as the polymers, fillers, lubricants, and coatings, and the testing/analytical equipment disclosed herein, are commercially available, unless otherwise noted. Oprozomib is not commercially available at the time of this writing.

This disclosure features gastro-retentive release pharmaceutical dosage forms (e.g., solid dosage forms, e.g., tablets) that are useful for the oral administration of oprozomib, or a pharmaceutically acceptable salt thereof, to a human or animal subject as well as methods of making and using the dosage forms.

In some embodiments, any one or more of the features described throughout the specification above can be combined with any one or more of the features described throughout the specification.

Table 1 illustrates two dosage forms (25 mg and 100 mg tablets) of the GR Bilayer Tablet and the amounts of active pharmaceutical agents and excipients (in weight percent and actual weight).

TABLE 1
Composition of GR Bilayer Tablet Dosage Form

| | 25 mg | | 100 mg | | | |
|---|---|---|---|---|---|---|
| | | Theoretical | | Theoretical | | |
| | Percentage | Quantity | Percentage | Quantity | | Reference to |
| Component | (w/w %) | (mg/tablet) | (w/w %) | (mg/tablet) | Function | Standard |

First Layer

| | | | | | | |
|---|---|---|---|---|---|---|
| Oprozomib | 4.17 | 25.0 | 16.67 | 100.0 | Active | In-house |
| | | | | | | specification |
| Microcrystalline | 61.83 | 371.0 | 49.33 | 296.0 | Filler | PhEur, |
| cellulose | | | | | | USP/NF, JP |
| PolyOx ® WSR | 12.00 | 72.0 | 8.00 | 48.0 | First | NF |
| 1105 LEO | | | | | swellable | |
| | | | | | polymer | |
| Magnesium | 0.50 | 3.0 | 0.50 | 3.0 | Lubricant | PhEur, |
| stearate (non- | | | | | | USP/NF, JP |
| bovine) | | | | | | |

Second Layer

| | | | | | | |
|---|---|---|---|---|---|---|
| PolyOx ® WSR | 21.00 | 126.0 | 25.00 | 150.0 | Second | NF |
| 303 LEO | | | | | swellable | |
| | | | | | polymer | |
| Magnesium | 0.50 | 3.0 | 0.50 | 3.0 | Lubricant | PhEur, |
| stearate (non- | | | | | | USP/NF, JP |
| bovine) | | | | | | |
| Core Tablet | 100.00 | 600.0 | 100.00 | 600.0 | | |
| Total | | | | | | |

Film Coating

| | | | | | | |
|---|---|---|---|---|---|---|
| Opadry II ® | 4.00 | 24.0 | 4.00 | 24.0 | Coating | Manufacturer |
| White | | | | | Material | Specification |
| (85F18422) | | | | | | |
| Purified water^{a} | — | — | — | — | Coating | USP |
| | | | | | Solvent | |

^{a}Removed during the drying process

Table 1a illustrates two dosage forms (150 mg and 200 mg tablets) of the GR Bilayer Tablet and the amounts of active pharmaceutical agents and excipients (in weight percent and actual weight).

TABLE 1a
Composition of GR Bilayer Tablet Dosage Forms

| | 150 mg | | 200 mg | | | |
|---|---|---|---|---|---|---|
| | | Theoretical | | Theoretical | | |
| | Percentage | Quantity | Percentage | Quantity | | Reference to |
| Component | (w/w %) | (mg/tablet) | (w/w %) | (mg/tablet) | Function | Standard |

First Layer

| | | | | | | |
|---|---|---|---|---|---|---|
| Oprozomib | 20.00 | 150.0 | 24.24 | 200.0 | Active | In House |
| | | | | | | Specification |
| Microcrystalline | 48.53 | 364.0 | 48.03 | 396.2 | Filler | PhEur, |
| Cellulose | | | | | | USP/NF, JP |
| PolyOx ® WSR | 10.67 | 80.0 | 8.73 | 72.0 | First | NF |
| 1105 LEO | | | | | Swellable | |
| | | | | | Polymer | |
| Magnesium | 0.80 | 6.0 | 0.82 | 6.8 | Lubricant | PhEur, |
| stearate (non- | | | | | | USP/NF, JP |
| bovine) | | | | | | |

Second Layer

| | | | | | | |
|---|---|---|---|---|---|---|
| PolyOx ® WSR | 19.80 | 148.5 | 18.00 | 148.5 | Second | NF |
| 303 LEO | | | | | Swellable | |
| | | | | | Polymer | |
| Magnesium | 0.20 | 1.5 | 0.18 | 1.5 | Lubricant | PhEur, |
| stearate (non- | | | | | | USP/NF, JP |
| bovine) | | | | | | |
| Core Tablet | 100.00 | 750.0 | 100.00 | 825.0 | | |
| Total | | | | | | |

Film Coating

| | | | | | | |
|---|---|---|---|---|---|---|
| Opadry II ® | 4.00 | 30.0 | 4.00 | 33.0 | Coating | Manufacturer |
| White | | | | | Material | Specification |
| (85F18422) | | | | | | |
| Purified water^{a} | — | — | — | — | Coating | USP |
| | | | | | Solvent | |

^{a}Removed during the drying process

Opadry II® White (85F18422) coating is a well know coating material, commercially available from Colorcon, Inc, of West Point, Pa.

PolyOx® WSR 1105 LEO® and PolyOx® WSR 303 LEO® are well known water soluble polyethylene oxide polymer resins, commercially available from The Dow Chemical Company, Midland, Mich.

Magnesium stearate is commercially available from numerous vendors, such as Parchem fine and specialty chemicals, New Rochelle, N.Y.

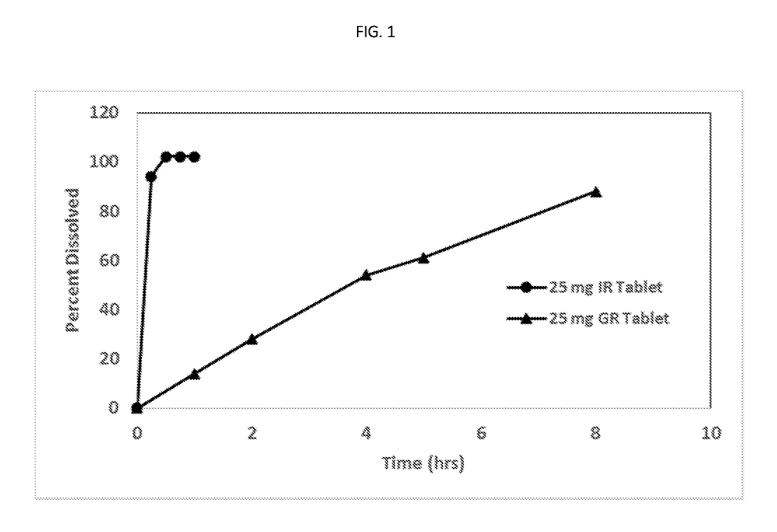

In another embodiment, the dosage forms described in Table 1 exhibited gastro-retentive release profiles, releasing equal to or greater than about 75% of oprozomib within about 8 hours or longer (see FIG. 1) under the dissolution conditions shown in Table 2:

TABLE 2

| | |
|---|---|
| Dissolution medium | About 0.01N Hydrochloric acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all rows |
| Residence Time | 2 hrs in first row |
| | 3 hrs in second row |
| | 3 hrs in third row |
| Sampling | Manual or automatic |
| Sampling Time | About 1, 2, 3, 4, 5, 7, 8 hours |
| Sampling Volume | About 5 mL |
| Screen size | 20 mesh |
| Filter | 5 um Titan 2 syringe filters (used new |
| | filters for each sampling time point) |
| UV Detection | 258 nm |
| Cell path length (for standard | 2 mm for 100 mg |
| UV spectrophotometer Cary ® | 10 mm for 25 mg |
| UV 50, Varian, Inc.)) | |

In another embodiment, the 150 mg dosage form described in Table 1a exhibited gastro-retentive release profiles, releasing equal to or greater than about 75% of oprozomib within about 8 hours or longer under the dissolution conditions shown in Table 2a:

TABLE 2a

| | |
|---|---|
| Dissolution medium | About 0.01N Hydrochloric |
| | acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all |
| | rows |
| Residence Time | 2 hrs in first row |
| | 2 hrs in second row |
| | 2 hrs in third row |
| | 2 hrs in fourth row |
| Sampling | Manual |
| Sampling Time | About 1, 2, 3, 4, 5, 7, 8 hours |
| Sampling Volume | About 5 mL |
| Screen size | 20 mesh |
| Filter | 5 um Titan 2 syringe filters |
| | (used new filters for each sampling |
| | time point) |
| UV Detection | 258 nm |
| Cell path length (for | 1 mm for 150 mg and 200 mg |
| standard UV | 2 mm for 100 mg |
| spectrophotometer Cary ® UV | 10 mm for 25 mg |
| 50, Varian, Inc.)) | |

The pharmaceutical dosage forms of oprozomib described herein provide a gastro-retentive modified release profile of oprozomib under the dissolution conditions listed in Table 2 or Table 2a and the UV detection above, releasing from about 40% to about 75% of the preferred dose at approximately 4 hours.

Accordingly, in one aspect, this disclosure features gastro-retentive modified release pharmaceutical dosage forms, which include an effective amount of oprozomib, or a pharmaceutically acceptable salt thereof, in which from about less than or equal to 40% of a preferred dose at approximately 1 hour as determined by UV under the dissolution conditions in Table 2 or Table 2a.

In another aspect, this disclosure features gastro-retentive release pharmaceutical dosage forms, which include an effective amount of oprozomib, or a pharmaceutically acceptable salt thereof; in which the dosage forms provide a reduced incidence or severity of one or more side effects (e.g., NVD).

In a further aspect, this disclosure features gastro-retentive modified release pharmaceutical dosage forms, which include an effective amount of oprozomib, or a pharmaceutically acceptable salt thereof; in which the dosage forms provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss).

As such, the dosage forms described herein can efficiently release oprozomib, e.g., to the stomach, duodenum and/or proximal part of the small intestine (e.g., jejunum), and do so over a modified period of time and, in some instances, with improved bioavailability, pharmacokinetic (PK) and/or pharmacodynamic (PD) parameters, thereby increasing the likelihood that oprozomib will be absorbed by the duodenum and jejunum prior to excretion and/or degradation of oprozomib. In a preferred embodiment, the dosage form increases the absorption of oprozomib in the duodenum and jejunum, leaving less of the drug to be present in the ileum and colon, which can cause GI tolerability issues. The dosage forms described herein can provide a reduced incidence or severity of one or more GI side effects (e.g., NVD). The present dosage forms can increase the GI tolerability of oprozomib, which can increase the likelihood of patient compliance with the dosage regimen.

In one embodiment, this disclosure features gastro-retentive release pharmaceutical dosage forms, which include an effective amount of oprozomib, or a pharmaceutically acceptable salt thereof; in which:
a. i) from about less than or equal to 40% of a preferred dose at approximately 1 hour;
   ii) from about 40% to about 75% of the preferred dose at approximately 4 hours; and
   iii) from about greater than or equal to 75% of the preferred dose at approximately 8 hours as determined by UV under the following dissolution conditions:

TABLE 2

| | |
|---|---|
| Dissolution medium | About 0.01N Hydrochloric acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all |
| | rows |
| Residence Time | 2 hrs in first row |
| | 3 hrs in second row |
| | 3 hrs in third row |
| Sampling | Manual or automatic |
| Sampling Time | About 1, 2, 3, 4, 5, 7, 8 hours |
| Sampling Volume | About 5 mL |
| Screen size | 20 mesh |
| Filter | 5 um Titan 2 syringe filters (used new |
| | filters for each sampling time point) |
| UV Detection | 258 nm |
| Cell path length (for standard | 2 mm for 100 mg |
| UV spectrophotometer Cary ® | 10 mm for 25 mg |
| UV 50, Varian, Inc.)) | |

and
b. the dosage forms provide a reduced incidence or severity of one or more side effects (e.g., NVD).

In certain embodiments, the dosage forms are in a form suitable for oral administration, e.g., a solid oral dosage form, e.g., a solid oral dosage form, e.g., a tablet, e.g., matrix tablet; e.g., matrix pellets; e.g., particulates filled into capsule; e.g., self-emulsified drug delivery systems (SEDDS).

In another embodiment, this disclosure features gastro-retentive release pharmaceutical dosage forms, which include an effective amount of oprozomib, or a pharmaceutically acceptable salt thereof; in which:
a. i) from about less than or equal to 40% of a preferred dose at approximately 1 hour;
   ii) from about 40% to about 75% of the preferred dose at approximately 4 hours; and
   iii) from about greater than or equal to 75% of the preferred dose at approximately 8 hours as determined by UV under the following dissolution conditions:

TABLE 2a

| | |
|---|---|
| Dissolution medium | About 0.01N Hydrochloric |
| | acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all |
| | rows |
| Residence Time | 2 hrs in first row |
| | 2 hrs in second row |
| | 2 hrs in third row |
| | 2 hrs in fourth row |
| Sampling | Manual |
| Sampling Time | About 1, 2, 3, 4, 5, 7, 8 hours |
| Sampling Volume | About 5 mL |
| Screen size | 20 mesh |
| Filter | 5 um Titan 2 syringe filters |
| | (used new filters for each sampling |
| | time point) |
| UV Detection | 258 nm |
| Cell path length (for | 1 mm for 150 mg and 200 mg |
| standard UV | 2 mm for 100 mg |
| spectrophotometer Cary ® UV | 10 mm for 25 mg |
| 50, Varian, Inc.)) | |

and
b. the dosage forms provide a reduced incidence or severity of one or more side effects (e.g., NVD).

In certain embodiments, the dosage forms are in a form suitable for oral administration, e.g., a solid oral dosage form, e.g., a solid oral dosage form, e.g., a tablet, e.g., matrix tablet; e.g., matrix pellets; e.g., particulates filled into capsule; e.g., self-emulsified drug delivery systems (SEDDS).

In one embodiment, this disclosure features gastro-retetentive, modified release pharmaceutical dosage forms, which include an effective amount of oprozomib, or a pharmaceutically acceptable salt thereof; in which:
a. i) from about less than or equal to 40% of a preferred dose at approximately 1 hour;
   ii) from about 40% to about 75% of the preferred dose at approximately 4 hours; and
   iii) from about greater than or equal to 75% of the preferred dose at approximately 8 hours, as determined by UV under the following dissolution conditions:

TABLE 2

| | |
|---|---|
| Dissolution medium | About 0.01N Hydrochloric acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all rows |
| Residence Time | 2 hrs in first row |
| | 3 hrs in second row |
| | 3 hrs in third row |
| Sampling | Manual or automatic |
| Sampling Time | About 1, 2, 3, 4, 5, 7, 8 hours |
| Sampling Volume | About 5 mL |
| Screen size | 20 mesh |
| Filter | 5 um Titan 2 syringe filters (used new |
| | filters for each sampling time point) |
| UV Detection | 258 nm |
| Cell path length (for standard | 2 mm for 100 mg |
| UV spectrophotometer Cary ® | 10 mm for 25 mg |
| UV 50, Varian, Inc.)) | |

b. the dosage forms provide a reduced incidence or severity of one or more side effects (e.g., NVD); and
c. the dosage forms provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss).

In another embodiment, this disclosure features gastro-retetentive, modified release pharmaceutical dosage forms, which include an effective amount of oprozomib, or a pharmaceutically acceptable salt thereof; in which:
a. i) from about less than or equal to 40% of a preferred dose at approximately 1 hour;
   ii) from about 40% to about 75% of the preferred dose at approximately 4 hours; and
   iii) from about greater than or equal to 75% of the preferred dose at approximately 8 hours, as determined by UV under the following dissolution conditions:

TABLE 2a

| | |
|---|---|
| Dissolution medium | About 0.01N Hydrochloric |
| | acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all |
| | rows |
| Residence Time | 2 hrs in first row |
| | 2 hrs in second row |
| | 2 hrs in third row |
| | 2 hrs in fourth row |
| Sampling | Manual |
| Sampling Time | About 1, 2, 3, 4, 5, 7, 8 hours |
| Sampling Volume | About 5 mL |
| Screen size | 20 mesh |
| Filter | 5 um Titan 2 syringe filters |
| | (used new filters for each sampling |
| | time point) |
| UV Detection | 258 nm |
| Cell path length (for | 1 mm for 150 mg and 200 mg |
| standard UV | 2 mm for 100 mg |
| spectrophotometer Cary ® UV | 10 mm for 25 mg |
| 50, Varian, Inc.)) | |

and
b. the dosage forms provide a reduced incidence or severity of one or more side effects (e.g., NVD); and
c. the dosage forms provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss).

An “effective amount” of oprozomib, or a pharmaceutically acceptable salt thereof, will vary from subject to subject, depending on species, age, and general condition of a subject, severity of the side effects or disorder, identity of the particular compound(s), mode of administration, and the like. As used herein, “an effective amount” refers to an amount of oprozomib, or a pharmaceutically acceptable salt thereof, that confers a therapeutic effect (e.g., controls, relieves, ameliorates, alleviates, or slows the progression of); or prevents (e.g., delays the onset of or reduces the risk of developing) a disease, disorder, or condition or symptoms thereof on the treated subject. The therapeutic effect may be objective (i.e., measurable by some test or marker) or subjective (i.e., subject gives an indication of or feels an effect).

In one aspect, methods for treating cancer (e.g., multiple myeloma, e.g., multiple myeloma that is relapsed and/or refractory; e.g., Waldenström's macroglobulinemia; e.g., myelodysplastic syndromes; e.g., chronic lymphocytic leukemia; e.g., plasma cell leukemia; e.g., hepatocellular cancer; e.g., mantle cell leukemia) in a patient are featured, which include administering to the patient a dosage form as described anywhere herein.

In another aspect, methods for treating autoimmune disease in a patient are featured, which include administering to the patient a dosage form as described anywhere herein.

In another aspect, methods for treating graft or transplant-related condition in a patient are featured, which include administering to the patient a dosage form as described anywhere herein.

In another aspect, methods for treating neurodegenerative disease in a patient are featured, which include administering to the patient a dosage form as described anywhere herein.

In another aspect, methods for treating fibrotic-associated condition in a patient are featured, which include administering to the patient a dosage form as described anywhere herein.

In another aspect, methods for treating ischemic-related condition in a patient are featured, which include administering to the patient a dosage form as described anywhere herein.

In another aspect, methods for treating an infection in a patient are featured, which include administering to the patient a dosage form as described anywhere herein.

In another aspect, methods for treating disease associated with bone loss in a patient are featured, which include administering to the patient a dosage form as described anywhere herein.

In one aspect, methods of preparing the dosage forms described herein are featured, comprises steps such as:

Step 1. Screen oprozomib and microcrystalline cellulose using a metal sieve.

Step 2. Blend the screened components with PolyOx®WSR 1105 LEO polymer in a suitable blender.

Step 3. Blend pre-screened magnesium stearate with materials from Step 2 in a suitable blender.

Step 4. Roller compact the blend into ribbons and subsequently mill the ribbons in the roller compactor equipped with a mill.

Step 5. Blend pre-screened magnesium stearate with PolyOx® WSR 303 LEO polymer in a suitable blender.

Step 6. Compress granules from Step 4 and blend from Step 5 using a rotary bilayer tablet press.

The tablet appearance, weight, and thickness are monitored throughout the compression process.

In another aspect, dosage forms prepared by the methods described herein are featured e.g., by granulation, e.g., wet granulation (e.g., foam granulation, spray drying, lyophilization), direct compression, dry granulation (e.g., slugging, roller compaction), fluid bed granulation, emulsification, extrusion spheronization, hot melt extrusion, pelletization, drug layering, coating.

The dosage form can provide a reduced incidence or severity of one or more side effects (e.g., nausea/vomiting (NVD)).

An embodiment of the present invention discloses a gastro-retentive modified release oral drug dosage form for releasing a sparingly soluble active pharmaceutical agent into the stomach, duodenum and/or upper small intestine of a patient. The drug dosage form comprises a first layer and a second layer; and an active pharmaceutical agent dispersed within at least one of the first layer and the second layer. The first layer further comprises a first swellable polymer and the second layer comprises a second swellable polymer.

The first swellable polymer swells via imbibition of water from gastric fluid to promote gastric retention in the stomach of the patient and sustain the release of the sparingly soluble active pharmaceutical agent. The second swellable polymer also swells via imbibition of water from gastric fluid to promote gastric retention in the stomach of the patient. Each first and second swellable polymers gradually erodes over a time period of hours, the erosion commencing upon contact with the gastric fluid, and wherein the erosion releases the sparingly soluble active pharmaceutical agent to the stomach, duodenum and/or upper small intestine of the patient, as a result of the erosion at a rate corresponding to the time period.

In another embodiment, the first and second swellable polymers each comprise polyethylene oxide and the sparingly soluble active pharmaceutical agent is oprozomib or a pharmaceutically acceptable salt thereof.

In another embodiment, the sparingly soluble active pharmaceutical agent within the first swellable polymer and the second swellable polymer.

In another embodiment, the oprozomib is dispersed within the first swellable polymer only.

In another embodiment, the oprozomib is dispersed within the second swellable polymer only.

In another embodiment, the first swellable polymer decreases the release rate of the pharmaceutical agent.

In another embodiment, the first and second swellable polymers exhibit different swelling and erosion rates.

In another embodiment, the second swellable polymer swells at a faster rate and erodes at a slower rate.

The dosage form can further include one or more fillers. The one or more fillers can be selected from microcrystalline cellulose, lactose monohydrate, dibasic calcium phosphate (“DCP”), sucrose, glucose, mannitol, and sorbitol.

The dosage form can optionally include one or more wetting agents (e.g., sodium laurel sulfate). Wetting agents can include surfactants or other surface active agents.

The dosage form can further include one or more lubricants (e.g., magnesium stearate).

The dosage form can further include one or more fillers (e.g., croscarmellose sodium).

The dosage form can further include one or more coatings (e.g., Opadry White (85F18422)).

The dosage form can be prepared by dry granulation, wet granulation or direct compaction.

The dosage form can include:

TABLE 3

| Component | Weight percent |
|---|---|
| Oprozomib, or a pharmaceutically | about 2.00 to about 50.00 |
| acceptable salt thereof | |
| One or more fillers | about 5.00 to about 90.00 |
| A first swellable polymer | about 2.00 to about 75.00 |
| A second swellable polymer | about 2.00 to about 75.00 |
| One or more lubricants | about 0.25 or 0.50 to about 5.00 |
| One or more coatings | about 1.00 to about 10.00 |

For example, the dosage form can include:

TABLE 4

| Component | Weight percent |
|---|---|
| Oprozomib, or a pharmaceutically | About 4.00 to about 17.00 |
| acceptable salt thereof | |
| One or more fillers | About 40.00 to about 65.00 |
| A first swellable polymer | About 5.00 to about 30.00 |
| A second swellable polymer | About 20.00 to about 30.00 |
| One or more lubricants | About 0.5 |
| One or more coatings | About 3.0 to about 5.00 |

The tablet can have a thickness of from about 2.5 millimeters to about 4.0 millimeters.

The tablet can have a thickness of from about 3.0 millimeters to about 3.77 millimeters, e.g., about 3.04 millimeters; e.g., about 3.75 millimeters.

The tablet can have a hardness of from about 1.00 to about 25.00 kilopond (“kp”), e.g., from about 5.00 to about 20.00 kp; from about 10.00 to about 20.00 kp; and from about 15.00 to about 20.00 kp.

In another embodiment, the dosage form can comprise the following Table 4a components and weight percent ranges:

TABLE 4a
High Strength Tablets from 100 mg to 200 mg

| Component | Weight percent |
|---|---|
| Oprozomib, or a pharmaceutically | About 12.00 to about 40.00 |
| acceptable salt thereof | |
| One or more fillers | About 30.00 to about 65.00 |
| A first swellable polymer | About 5.00 to about 20.00 |
| A second swellable polymer | About 16.00 to about 30.00 |
| One or more lubricants | About 0.25 to about 1.50 |
| One or more coatings | About 3.00 to about 6.00 |

The high strength tablet can have a thickness of from about 4.0 millimeters to about 8.0 millimeters.

The tablet can have a thickness of from about 5.0 millimeters to about 7.62 millimeters, e.g., about 5.59 millimeters; e.g., about 7.37 millimeters.

The tablet can have a hardness of from about 1.00 to about 30.00 kilopond (“kp”), e.g., from about 5.00 to about 20.00 kp; from about 10.00 to about 20.00 kp; and from about 15.00 to about 20.00 kp.

The dosage form can be stable upon actual or simulated storage at 40° C./75% relative humidity (RH) for at least 1 month.

Dosage Form Components

Typically, the dosage form components are commercially available, unless otherwise noted.

Typically, the dosage forms described herein include one or more components that modify the rate at which oprozomib is released from the dosage form into the body. The one or more components can be present in the core of the dosage form and/or in a coating(s) that surrounds the dosage forms.

In some embodiments, the one or more components that modify the rate at which oprozomib is released from the dosage form into the body can be one or more pharmaceutically acceptable polymers.

In some embodiments, the one or more pharmaceutically acceptable polymers can be any hydrophilic or lipophilic based controlled release polymers and excipients derived from natural, synthetic and/or semi-synthetic sources.

In certain embodiments, the one or more pharmaceutically acceptable polymers can be one or more matrix-forming polymers, e.g., one or more hydrophilic matrix-forming polymers.

In certain embodiments, the one or more pharmaceutically acceptable polymers can be Nonionic homo-polymers of ethylene oxide such as polyethylene oxide (e.g. Polyox WSR N-12K, WSR N-60K, WSR-301, WSR-coagulant, WSR-303 LEO, WSR-1105 LEO, WSR-308).

In certain embodiments, the one or more pharmaceutically acceptable polymers can be a mixture of one or more matrix-forming polymers, e.g., one or more hydrophilic matrix-forming polymers, and one or more insoluble polymers, e.g., one or more ammoniomethacrylate copolymers.

In certain embodiments, the one or more hydrophilic matrix-forming polymers or polymers is hydroxy propyl methylcellulose (“HPMC”). In some embodiments, the one or more ammoniomethacrylate copolymer is Eudragit® (Rohm and Haas, Phila., Pa.).

In some embodiments, the dosage forms described herein can include one or more of the following:
Non-ionic soluble cellulose ethers, such as hydroxypropyl methylcellulose (HPMC, e.g., Methocel® K100LV, K4M, K15M, K100M (Dow Chemical); Benecel® MP843, MP 814, MP844 (Ashland); Metolose® 100, 4000, 15000 and 100000 SR (Shin-Etsu Chemical Co., Totowa, N.J.), hydroxypropyl cellulose (HPC, e.g., Klucel® GXF, MXF, HXF (Ashland)), hydroxyethyl cellulose (HEC, e.g., Natrosol® 250 HHX, HX, M, G (Ashland)),) with various degrees of substitutions and viscosity grades
Water-soluble natural gums of polysaccharides of natural origin, such as xanthan gum, alginate, and locust bean gum
Water swellable, but insoluble, high molecular weight homo-polymers and copolymers of acrylic acid chemically cross-linked with polyalkenyl alcohols with varying degree of cross-linking or particle size (Carbopol® 71G NF, 971P, 974P, 934P (Lubrizol Corporation, Wickliffe, Ohio)
Polyvinyl acetate and povidone mixtures (Kollidon® SR, BASF)
Cross-linked high amylose starch
Ionic methacrylate copolymers (Eudragit® L30D, FS30D)
Fatty acids, fatty acid esters, mono-, di- and tri-glycerides of fatty acids, fatty alcohols, waxes of natural and synthetic origins with differing melting points e.g., stearic acid, lauryl, cetyl or cetostearyl alcohol, glyceryl behenate, carnauba wax, beeswax, candelila wax, microcrystalline wax and low molecular weight polyethylene.
Insoluble polymers include ammoniomethacrylate copolymers (Eudragit® RL100, PO, RS100, PO, NE-30D, RL-30D, RS-30D, RL PO), ethyl cellulose (Ethocel® (Dow), Surelease® (Colorcon), Aquacoat® ECD (FMC)), cellulose acetate (CA-398-10), cellulose acetate butyrate (CAB-381-20), cellulose acetate propionate (CAP-482-20), cellulose acetate phthalate (Aquacoat® CPD), polyvinylacetate (Kollicoat® (BASF)).
Effervescent components include sodium bicarbonate, citric acid, stearic acid, and combinations thereof.

In certain embodiments, one or more pharmaceutically acceptable polymers or polymer resins are utilized as a first swellable polymer, a second swellable polymer and fillers.

In certain embodiments, one or more pharmaceutically acceptable polymers or polymer resins are polyethylene oxide, such as PolyOx WSR 1105 LEO® and PolyOx® WSR 303 LEO®.

Gastro-Retentive Modified Release Bilayer Tablet

The dosage form can be in a form that is suitable for oral administration, such as a bilayer tablet, wherein a first layer comprises a first swellable polymer and a second layer comprises a second swellable polymer of the bilayer tablet. The active pharmaceutical agent may be dispersed within only one of the swellable polymers or both swellable polymers.

Preferably, the active pharmaceutical agent, oprozomib is dispersed within the first swellable polymer with a lower molecular weight. The first swellable polymer swells at a slower rate and erodes at a faster rate than the second swellable polymer, thereby delaying the release of the active pharmaceutical agent, preferably oprozomib, in the stomach, duodenum and/or jejunem. The second swellable polymer, with a higher molecular weight than the first swellable polymer, swells at a faster rate and erodes at a slower rate to maintain the presence of the tablet in the stomach, duodenum and/or jejunem.

Oprozomib has an absorption window in the gastrointestinal tract where oprozomib is well absorbed in the duodenum and jujenum regions of the GI tract. In addition, oprozomib exhibits adverse GI events, such as NVD, which are attributed to a local GI effect. The swelling properties of the polymers are important in that they allow the dosage forms to be retained in the stomach where they effectively deliver drugs on a continuous basis to the stomach, duodenum, jejunum, and upper sections of the small intestine where absorption is efficient. The beneficial properties of the swelling polymers include protecting drugs against the degradative environment of the G.I. tract, overcoming a too rapid drug release rate due to high drug solubility, or targeting drugs to specific areas within the G.I. tract.

Compared to known practices, the present invention GR modified release tablet utilizes one or more different grades, preferably two, of polyethylene oxide to develop a bilayer gastroretentive (GR) tablet. Polyethylene oxide, preferably Poly OX® WSR 1105 LEO®, commercially available from The Dow Chemical Company, comprises a first swellable polymer, regulating the modified release of the active pharmaceutical agent, preferably oprozomib, so that it releases:
a. from about less than or equal to 40% of a preferred dose at approximately 1 hour;
b. from about 40% to about 75% of the preferred dose at approximately 4 hours; and
c. from about greater than or equal to 75% of the preferred dose at approximately 8 hours.

A different grade of polymer, preferably with a greater molecular weight than the first polymer, preferably a polyethylene oxide polymer, more preferably PolyOx® WSR 303 LEO® polymer, commercially available from The Dow Chemical Company, is used in the second swellable polymer, promoting the swelling of the tablet to a preferred size and eroding of the tablet to a preferred slower rate which allows the tablet to maintain at least a minimum size and remain in the stomach for an extended period of time with the administration of food, before, during and after the ingestion of the tablet. The addition of the second swellable polymer serves to promote the extension of time the tablet remains in the stomach with food.

In some embodiments, the dosage forms can include from about 2.00 weight percent to about 80.00 weight percent of a first swellable polymer; from about 5.00 weight percent to about 75.00 weight percent of a first swellable polymer; from about 10.00 weight percent to about 60.00 weight percent of a first swellable polymer, from about 10.00 weight percent to about 40.00 weight percent of a first swellable polymer, from about 20.00 weight percent to about 30.00 weight percent of a first swellable polymer.

In some embodiments, the dosage forms can include from about 2.00 weight percent to about 80.00 weight percent of a second swellable polymer; from about 5.00 weight percent to about 75.00 weight percent of a second swellable polymer; from about 15.00 weight percent to about 50.00 weight percent of a second swellable polymer; from about 20.00 weight percent to about 40.00 weight percent of a second swellable polymer; from about 20.00 weight percent to about 30.00 weight percent of a second swellable polymer.

Oprozomib Oprozomib can be prepared, e.g., according to the synthetic route and procedures delineated in Example 1. As used herein, “oprozomib” without a modifier such as “in the form of a pharmaceutically acceptable salt” is intended to refer to the free-base form of oprozomib.

In some embodiments, the dosage forms include oprozomib.

In some embodiments, the dosage forms include oprozomib in the form of a pharmaceutically acceptable salt.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In certain embodiments, the dosage forms include amorphous oprozomib.

In certain embodiments, the dosage forms include one or more crystalline forms of oprozomib. An example of such a crystalline form of oprozomib is described in, e.g., US-2012-0077855, which is incorporated herein by reference in its entirety. Said crystalline form can include any one or more of the following features.

The crystalline form of oprozomib can have an X-ray powder diffraction pattern that includes one of the following characteristic peaks expressed in degrees 2θ: 9.4 (or about 9.4); 24.3 (or about 24.3); 11.1 (or about 11.1); or 15.3 (or about 15.3).

The crystalline form of oprozomib can have an X-ray powder diffraction pattern that includes any two, three or four of the following characteristic peaks: 9.4 (or about 9.4), 11.1 (or about 11.1), 15.3 (or about 15.3), and 24.3 (or about 24.3) (each expressed in degrees 2θ).

The crystalline form of oprozomib can have an X-ray powder diffraction pattern that includes the characteristic peak expressed in degrees 2θ at 9.4 (or about 9.4) and one of the following characteristic peaks: (i) the characteristic peak expressed in degrees 2θ at 24.3 (or about 24.3); or (ii) the characteristic peak expressed in degrees 2θ at 11.1 (or about 11.1); or (iii) the characteristic peak expressed in degrees 2θ at 15.3 (or about 15.3).

The crystalline form of oprozomib can have an X-ray powder diffraction pattern that includes the characteristic peaks expressed in degrees 2θ at 9.4 (or about 9.4), 11.1 (or about 11.1), and 24.3 (or about 24.3).

The crystalline form of oprozomib can have an X-ray powder diffraction pattern that includes the characteristic peaks expressed in degrees 2θ at 9.4 (or about 9.4), 11.1 (or about 11.1), 15.3 (or about 15.3), and 24.3 (or about 24.3).

The X-ray powder diffraction pattern of the crystalline form of oprozomib can also include one (or more) lower intensity characteristic peaks. The relative intensities of these additional peak(s) are, in general, lower than the relative intensities associated with the four characteristic peaks described above.

The crystalline form of oprozomib can have an X-ray powder diffraction pattern that includes characteristic peaks expressed in degrees 2θ at 9.4 (or about 9.4), 11.1 (or about 11.1), 15.3 (or about 15.3), 22.3 (or about 22.3), and 24.3 (or about 24.3).

The crystalline form of oprozomib can have an X-ray powder diffraction pattern that includes characteristic peaks expressed in degrees 2θ at 9.4 (or about 9.4), 11.1 (or about 11.1), 12.7 (or about 12.7), 15.3 (or about 15.3), 22.3 (or about 22.3), 24.3 (or about 24.3), and 28.3 (or about 28.3).

The crystalline form of oprozomib can have an X-ray powder diffraction pattern that includes characteristic peaks expressed in degrees 2θ at 9.4 (or about 9.4), 11.1 (or about 11.1), 12.7 (or about 12.7), 15.3 (or about 15.3), 20.9 (or about 20.9), 21.8 (or about 21.8), 22.3 (or about 22.3), 24.3 (or about 24.3), 28.3 (or about 28.3), 29.0 (or about 29.0), 29.7 (or about 29.7), and 30.5 (or about 30.5).

The crystalline form of oprozomib can have an X-ray powder diffraction pattern that includes characteristic peaks expressed in degrees 2θ at 8.9 (or about 8.9); 9.4 (or about 9.4); 9.8 (or about 9.8); 10.6 (or about 10.6); 11.1 (or about 11.1); 12.7 (or about 12.7); 15.3 (or about 15.3); 17.7 (or about 17.7); 19.0 (or about 19.0); 20.6 (or about 20.6); 20.9 (or about 20.9); 21.6 (or about 21.6); 21.8 (or about 21.8); 22.3 (or about 22.3); 22.8 (or about 22.8); 24.3 (or about 24.3); 24.7 (or about 24.7); 26.0 (or about 26.0); 26.4 (or about 26.4); 28.3 (or about 28.3); 29.0 (or about 29.0); 29.7 (or about 29.7); 30.2 (or about 30.2); 30.5 (or about 30.5); 30.8 (or about 30.8); 32.1 (or about 32.1); 33.7 (or about 33.7); 34.5 (or about 34.5); 35.1 (or about 35.1); 35.3 (or about 35.3); 37.9 (or about 37.9); and 38.5 (or about 38.5).

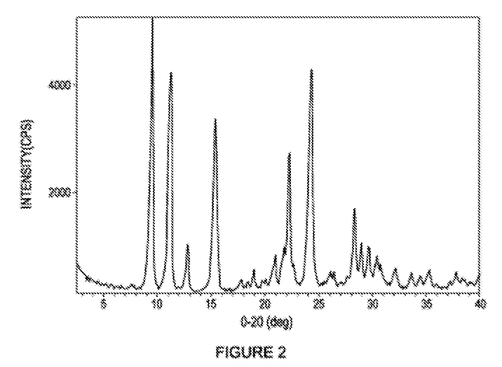

The crystalline form of oprozomib can have an X-ray powder diffraction pattern that is substantially the same as that shown (substantially as shown) in FIG. 2. The term “about” when used in conjunction with defining a position of a characteristic peak in an X-ray powder diffraction pattern is intended to mean the stated degree 2θ value ±0.2 degrees 2θ.

In some embodiments, the location(s) of characteristic peak(s) can be expressed to the nearest tenth (0.1) of a degree 2θ.

The crystalline form of oprozomib can also have one or more of the following characteristic features.

The crystalline form of oprozomib can have a differential scanning calorimetry pattern that includes a melting onset of about 140° C.

The crystalline form of oprozomib can have a differential scanning calorimetry pattern that includes a sharp endothermic maximum at about 147° C.

The crystalline form of oprozomib can have a differential scanning calorimetry pattern that includes a melting onset of about 140° C. and a sharp endothermic maximum at about 147° C.

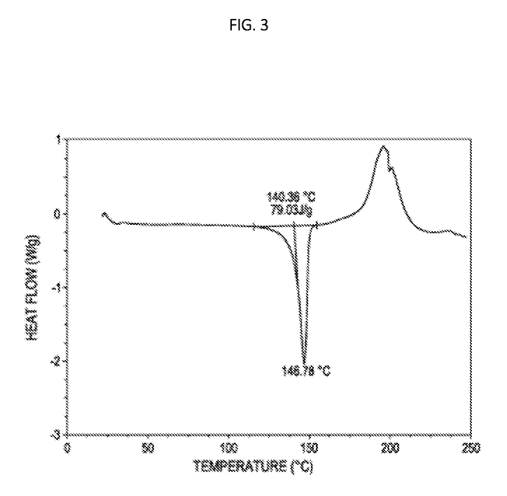

The crystalline form of oprozomib can have a differential scanning calorimetry pattern that is substantially the same as that shown (substantially as shown) in FIG. 3.

The crystalline form of oprozomib can have a melting point from about 140 to about 155° C. (e.g., from about 145 to about 150° C.).

The crystalline form of oprozomib can exhibit from 0.0 to 0.3% weight loss in the temperature range of 25 to 125° C.

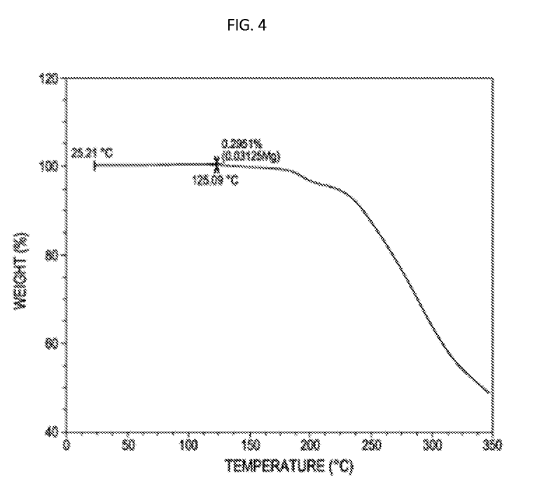

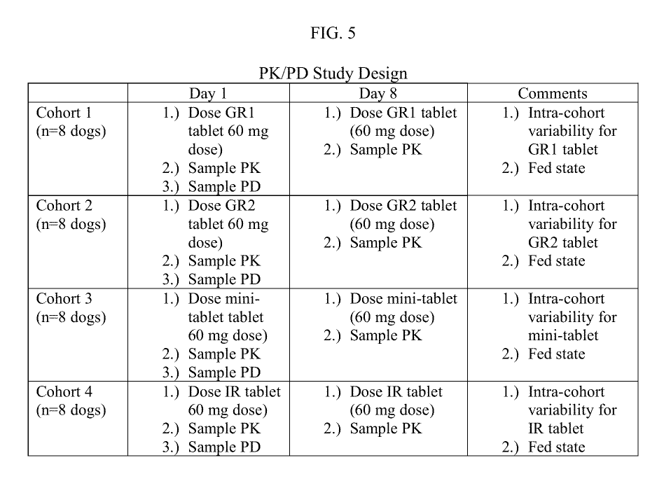

The crystalline form of oprozomib can have a thermogravimetric analysis pattern that is substantially the same as that shown (substantially as shown) in FIG. 4.

In certain embodiments, the dosage forms include both amorphous oprozomib and one or more crystalline forms of oprozomib as described anywhere herein.

In some embodiments, the dosage forms include oprozomib in the form of a pharmaceutically acceptable salt.

In certain embodiments, the dosage forms include amorphous oprozomib in the form of a pharmaceutically acceptable salt.

In certain embodiments, the dosage forms include one or more crystalline forms of oprozomib in the form of a pharmaceutically acceptable salt.

In some embodiments, the dosage forms include both oprozomib and oprozomib in the form of a pharmaceutically acceptable salt. These embodiments can include any combination of amorphous oprozomib, one or more crystalline forms of oprozomib, amorphous oprozomib in the form of a pharmaceutically acceptable salt, and one or more crystalline forms of oprozomib in the form of a pharmaceutically acceptable salt, each as described anywhere herein.

The dosage form can include from about 2.00 weight percent to about 50.00 weight percent of oprozomib, or a pharmaceutically acceptable salt thereof (e.g., from about 3.00 weight percent to about 25.00 weight percent of oprozomib, or a pharmaceutically acceptable salt thereof, from about 4.00 weight percent to about 20.00 weight percent of oprozomib, or a pharmaceutically acceptable salt thereof e.g., from about 4.00 weight percent to about 17.00 weight percent of oprozomib, or a pharmaceutically acceptable salt thereof, such as about 4.17 weight percent to about 16.67 weight percent of oprozomib, or a pharmaceutically acceptable salt thereof).

The dosage form can include from about 10.00 mg to about 600.00 mg of oprozomib, or a pharmaceutically acceptable salt thereof, from about 20.00 mg to about 500.00 mg of oprozomib, or a pharmaceutically acceptable salt thereof, from about 20.00 mg to about 300.00 mg of oprozomib, or a pharmaceutically acceptable salt thereof, from about 25.00 mg to about 150.00 mg of oprozomib, or a pharmaceutically acceptable salt thereof, from about 25.00 mg to about 112.50 mg of oprozomib, or a pharmaceutically acceptable salt thereof, from about 25.00 mg to about 100.00 mg of oprozomib, or a pharmaceutically acceptable salt thereof, and from about 25.00 mg to about 50.00 mg of oprozomib, or a pharmaceutically acceptable salt thereof.

The dosage form can include from about 25.00 milligrams of oprozomib, or a pharmaceutically acceptable salt thereof about 50.00 milligrams of oprozomib, or a pharmaceutically acceptable salt thereof about 75.00 milligrams of oprozomib, about 100.00 milligrams of oprozomib, about 112.50 milligrams of oprozomib, about 150.00 milligrams of oprozomib, about 300.00 milligrams of oprozomib, or a pharmaceutically acceptable salt thereof.

The dosage form can include from about 4.00 weight percent to about 5.00 weight percent of oprozomib, or a pharmaceutically acceptable salt thereof and about 25.00 milligrams of oprozomib, or a pharmaceutically acceptable salt thereof.

The dosage form can include from about 16.00 weight percent to about 17.00 weight percent of oprozomib, or a pharmaceutically acceptable salt thereof and about 50.00 milligrams of oprozomib, or a pharmaceutically acceptable salt thereof.

The dosage form can include from about 4.17 weight percent of oprozomib, or a pharmaceutically acceptable salt thereof and about 25.00 milligrams of oprozomib, or a pharmaceutically acceptable salt thereof.

The dosage form can include from about 16.67 weight percent of oprozomib, or a pharmaceutically acceptable salt thereof and about 50.00 milligrams of oprozomib, or a pharmaceutically acceptable salt thereof. The dosage form can include from about 50 weight percent of oprozomib, or a pharmaceutically acceptable salt thereof and about 300.00 milligrams of oprozomib, or a pharmaceutically acceptable salt thereof.

The dosage form can include from about 50 weight percent of oprozomib, or a pharmaceutically acceptable salt thereof and about 600.00 milligrams of oprozomib, or a pharmaceutically acceptable salt thereof.

The oprozomib, or pharmaceutically acceptable salt thereof, can be a crystalline solid.

The oprozomib, or pharmaceutically acceptable salt thereof, can be an amorphous solid.

Non-Limiting Excipients

In some embodiments, the dosage forms further include one or more pharmaceutically acceptable excipients. Pharmaceutically acceptable excipients include any and all fillers, binders, surfactants (wetting agents), sugars, antioxidants, solubilizing or suspending agents, chelating agents, preservatives, colorants, buffering agents and/or lubricating agents, or combinations thereof, as suited to the particular dosage form desired and according to the judgment of the formulator. Remington's Pharmaceutical Sciences, Sixteenth Edition, E. W. Martin (Mack Publishing Co., Easton, Pa., 1980) discloses various pharmaceutically acceptable excipients used in preparing pharmaceutically acceptable dosage forms and known techniques for the preparation thereof. In general, the weight percent of the one or more pharmaceutically acceptable excipients (e.g., one or more fillers) varies with the weight percent and/or strength or purity of the oprozomib, or a pharmaceutically acceptable salt thereof; and, in some instances, the amount of oprozomib, or a pharmaceutically acceptable salt thereof, and the amount(s) of one or more other dosage form components, e.g., a polymer component, e.g., HPMC.

In some embodiments, the dosage forms include one or more fillers. As used herein, the term “filler” refers to any pharmaceutically acceptable agent that is added to a pharmaceutical preparation or dosage form to increase the weight or size (e.g., “bulk”) of the pharmaceutical preparation or dosage form.

Non-limiting examples of fillers include starch (e.g., corn starch or potato starch), Pregelatinized Starch (Starch 1500), Microcrystalline Cellulose, Modified (crosslinked) Starches (e.g.; Sodium Carboxymethyl Starch), Cross-linked polyvinylpyrrolidone, Modified (crosslinked) Cellulose (i.e. Ac-Di-Sol (Accelerates Dissolution), croscarmellose sodium Nymcel), crosslinked alginic acid (such as Alginic acid NF and Satialgine®), natural super fillers (such as soy polysaccharides or Emcosoy®), croscarmellose sodium, calcium silicate, lactose monohydrate, dibasic calcium phosphate (“DCP”), sucrose, glucose, mannitol, and sorbitol. Preferred fillers include microcrystalline cellulose and lactose monohydrate.

In some embodiments, the dosage forms can include two or more fillers. For example, the fillers can include microcrystalline cellulose (e.g., Avicel® PH101 or Avicel® PH102 (Sigma Aldrich, St. Louis, Mo.)) and lactose monohydrate (e.g., Lactose 312® or Lactose 316® (Foremost Farms, Barbaroo, Wis.)).

In some embodiments, the dosage forms can include one or more fillers from about 2.00 to about 80.00 weight percent; from about 15.00 to about 75.00 weight percent; from about 25.00 to about 65.00 weight percent; from about 35.00 to about 65.00 weight percent; from about 40.00 to about 65.00 weight percent. For example, the dosage forms can include about 49 weight percent of the one or more fillers or about 62 weight percent of the one or more fillers or about 55 weight percent of the one or more fillers.

In some embodiments, the dosage forms include one or more lubricants. As used herein, the term “lubricant” refers to a pharmaceutically acceptable substance that reduces the friction associated with tablet ejection between the walls of the tablet and the walls of a cavity used to form the tablet (see The Theory and Practice of Industrial Pharmacy, Third Edition. Leon Lachman, Herbert Lieberman, and Joseph Kanig, editors. Lea & Febiger, Philadelphia. 1986, page 328).

Suitable lubricants include magnesium stearate; metal stearates, glyceryl behenate, sodium stearyl fumarate, hydrogenated vegetable oils, or fatty acids Sigma Aldrich). An exemplary lubricant is magnesium stearate.

In some embodiments, the dosage forms can include from about 0.10 weight percent to about 2.00 weight percent (e.g., from 0.50 weight percent to about 1.20 weight percent, e.g., about 0.50 weight percent) of a lubricant.

In some embodiments, the dosage forms include materials, which are both lubricated (can function as a lubricant) and can function as a filler (e.g., siliconized MCC).

In some embodiments, such as when the dosage form is comprised of a tablet or capsule form, the dosage form can further include one or more coatings (e.g., Opadry II® White (85F18422) or another color). In some embodiments, the dosage forms can include from about 1.00 weight percent to about 10.00 weight percent (e.g., from 0.50 weight percent to about 5.00 weight percent, e.g., about 4.00 weight percent) of a coating.

### Non-Limiting Combinations of Dosage Form Components

In some embodiments, the dosage forms comprise:
a. oprozomib, or a pharmaceutically acceptable salt thereof; and
b. one or more components that modify the rate at which oprozomib is released from the dosage form into the body (e.g., one or more swellable polymers).

In some embodiments, the dosage forms described above include:
a. oprozomib, or a pharmaceutically acceptable salt thereof;
b. one or more components that modify the rate at which oprozomib is released from the dosage form into the body (e.g., one or more swellable polymers and mixtures thereof); and
c. one or more pharmaceutically acceptable excipients (e.g., one or more fillers and/or one or more fillers and/or one or more lubricants).

For example, the dosage forms described above can comprise:

TABLE 5

| Component | Weight percent |
|---|---|
| Oprozomib, or a | From about 2.00 to about 50.00 (e.g., |
| pharmaceutically acceptable | about 3.00 to about 25.00, e.g., about |
| salt thereof | 4.00 to about 20.00, e.g., about 4.00 to |
| | about 17.00, e.g., about 4.17 to about |
| | 16.67.) |
| One or more fillers | From about 2.00 to about 80.00; from |
| | about 15.00 to about 75.00; from about |
| | 25.00 to about 65.00; from about 35.00 |
| | to about 65.00; from about 40.00 to |
| | about 65.00. |
| First swellable polymer | From about 5.00 to about 95.00 (e.g., |
| | from about 5.00 to about 50.00, e.g., |
| | about 5.00 to about 25.00, e.g., about |
| | 6.00 to about 15.00, e.g., about 8.00 to |
| | about 12.00) |
| Second swellable polymer | From about 2.00 to about 80.00; from |
| | about 10.00 to about 65.00; from about |
| | 15.00 to about 50.00; from about 20.00 |
| | to about 30.00. |
| One or more lubricants | From about 0.10 to about 2.00; from |
| | about 0.50 to about 1.20; about 0.50. |
| One or more coatings | From about 0.01 to about 10.00 (e.g., |
| | about 3.0 to about 5.0, e,g., about 4.0). |

### In Another Embodiment, the Dosage Form can Comprise:

TABLE 5a

| Component | Weight percent |
|---|---|
| Oprozomib, or a | From about 2.00 to about 50.00 (e.g., |
| pharmaceutically acceptable | about 12.00 to about 40.00, e.g., about |
| salt thereof | 15.00 to about 34.00, e.g., about 17.00 |
| | to about 30.00, e.g., about 16.67 to |
| | about 24.24) |
| One or more fillers | From about 2.00 to about 80.00; from |
| | about 15.00 to about 75.00; from about |
| | 30.00 to about 65.00; from about 33.00 |
| | to about 50.00; from about 40.00 to |
| | about 49.00. |
| First swellable polymer | From about 5.00 to about 95.00 (e.g., |
| | from about 5.00 to about 50.00, e.g., |
| | about 5.00 to about 25.00, e.g., about |
| | 6.00 to about 15.00, e.g., about 8.00 to |
| | about 12.00) |
| Second swellable polymer | From about 2.00 to about 80.00; from |
| | about 10.00 to about 65.00; from about |
| | 15.00 to about 50.00; from about 18.00 |
| | to about 30.00. |
| One or more lubricants | From about 0.10 to about 2.00; from |
| | about 0.50 to about 1.50; about 0.50; |
| | about 1.0 |
| One or more coatings | From about 0.01 to about 10.00 (e.g., |
| | about 3.0 to about 5.0, e,g., about 4.0). |

Dosage Forms

In general, the gastro-retentive release pharmaceutical dosage forms of oprozomib described herein can be prepared in a form that is suitable for oral administration, which is among the preferred routes for administration of pharmaceuticals since this route is generally convenient and acceptable to patients. In certain embodiments, oral administration of the dosage forms is preferred, and the dosage forms can be in any form that is suitable for oral administration (e.g., any conventional oral dosage forms including, but not limited to, solid dosage forms such as a tablet, a pill, a hard or soft capsule, a dragee, a lozenge, a cachet, a sachet, a powder (e.g., dispensable powder), granules, a matrix tablet, e.g., matrix pellets, e.g., particulates filled into capsule, e.g., self-emulsified drug delivery systems (SEDDS)); and liquid preparations such as syrups, slurries, gels, pellets, particulates, elixirs, emulsions and aqueous suspensions, dispersions, solutions, and concentrated drops, or any other form reasonably adapted for oral administration).

In some embodiments, the dosage forms can be in the form of a discrete, solid oral dosage unit (e.g. a capsule, a tablet, or a dragee) containing a predetermined amount of any one or more of the components described herein.

In some embodiments, the dosage forms can be in the form of a tablet. Such forms can be shaped and dimensioned as desired. In certain embodiments, the dosage forms can be in the form of a tablet that is capsule-shaped. In some embodiments, the tablet can be a modified capsule shaped core tablet.

In certain embodiments, the dosage forms can be in the form of a tablet having a thickness of from about 2.5 to about 12.0 millimeters (mm) (e.g., from about 2.0 to about 4.0 millimeters, from about 3.0 to about 3.8 millimeters, from about 3.0 millimeters to about 3.77 millimeters, from about 4.0 millimeters to about 8.0 millimeters, from about 5.0 millimeters to about 7.62 millimeters, e.g., about 5.59 millimeters; e.g., about 7.37 millimeters).

In certain embodiments, the dosage forms can be in the form of a “compressed tablet,” which as used herein refers to a plain, uncoated tablet for oral ingestion. Compressed tablets are typically prepared by a single compression or by pre-compaction tapping followed by a final compression (e.g., using a Carver press, rotary press, single station tablet press (Carver, Inc., Wabash, Ind.)). The tablets can be scored, printed, and/or debossed or embossed with desired identifier markings.

The dosage form can be in a bilayer tablet, wherein a first swellable polymer comprises a first layer and a second swellable polymer comprises a second layer of the bilayer tablet. The active pharmaceutical agent may be dispersed within only swellable polymer or both swellable polymers.

In some embodiments, the bilayer tablets can have a hardness of from about 1.0 kp to about 50.0 kp (e.g., from about 3.0 kp to about 30.0 kp, from about 5.0 kp to 25.0 kp, from about 10.0 kp to about 20.0 kp, from about 16.0 kp to about 20.0 kp).

In certain embodiments, the tablet can be a coated tablet. As a further example, tablets can also be coated with a conventional coating material such as Opadry™ White 85F18422 (or another color). In some embodiments, the coating is present from about 1.00 to about 10.00 weight percent of the tablet. For example, the coating can be present at about 4.00 weight percent.

In certain embodiments, the weight of the tablet can be from about 5 milligrams to about 1,200 milligrams (e.g., about 60 milligrams to about 1,000 milligrams; from about 70 milligrams to about 600 milligrams; e.g., about 75 milligrams, about 78 milligrams, about 80 milligrams, about 100 milligrams, about 150 milligrams, about 156 milligrams, about 200 milligrams, about 250 milligrams, about 400 milligrams or about 450 milligrams).

In general, the dosage forms can be prepared by any suitable and conventional method of pharmacy known in the art, which includes the step of bringing into association any one or more of the components described herein. Methods of preparation can include one or a combination of methods including: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986).

In some embodiments, the dosage forms can be obtained, for example, by performing one or more of the following steps: (i) combining (e.g., uniformly and intimately admixing so as to disperse the active ingredient evenly throughout the dosage form, e.g., to facilitate subdivision of the dosage form into unit dosage forms) the active ingredient, surfactant(s), and any other component(s) described herein to provide a mixture; (ii) screening, sieving, grinding, and/or milling the resulting mixture; (iii) processing the mixture of granules, after adding suitable auxiliaries, if desired; (iv) shaping and optionally coating the product to obtain tablets or dragee cores; or (v) adding the processed dosage form to a vessel suitable for oral administration, such as a capsule.

In certain embodiments, the dosage forms can be prepared using wet granulation techniques known in the art, which can include the steps of milling and sieving of the ingredients, dry powder mixing, wet massing, granulation and final grinding. In some embodiments, the wet granulation techniques such as high shear granulation, fluid bed granulation, extrusion spheronization etc. can better accommodate the micronized active ingredients and can result in dosage forms having enhanced powder flow (for encapsulation) and dissolution properties.

In certain embodiments, the dosage forms can be prepared using dry granulation techniques known in the art, which can include the steps of milling and sieving of the ingredients, dry powder mixing, and final blending.

In certain embodiments, compressed tablets can be prepared by compressing, in a suitable machine, such as a roller compaction machine, the dosage form in a free-flowing form, such as a powder or granules. Molded tablets can be made by molding, in a suitable machine, the powdered dosage form moistened with an inert liquid diluent.

In some embodiments, the dosage forms provide a reduced incidence or severity of one or more side effects (e.g., NVD).

In some embodiments, the dosage forms provide a therapeutically effective plasma exposure of oprozomib resulting in near complete proteasome inhibition of target tissues e.g., effective to treat one or more of the disorders described herein (e.g., cancer, autoimmune disease, graft or transplant-related condition, neurodegenerative disease, fibrotic-associated condition, ischemic-related conditions, infection (viral, parasitic or prokaryotic) and diseases associated with bone loss).

In some embodiments, the dosage forms described herein can deliver oprozomib with time to peak plasma concentrations of from about 1 to about 8 hours, from about 4 to about 6 hours and about 6 hours (See FIGS. 6, 7, 10 and 10(a)) as determined in dogs; as such, the dosage forms described herein can efficiently release oprozomib, e.g., to the stomach and proximal part of the small intestine, and do so over an immediate period of time and, in some instances, with improved bioavailability, pharmacokinetic (PK) and/or pharmacodynamic (PD) parameters, thereby increasing the likelihood that oprozomib will be absorbed by the duodenum and jejunum prior to excretion and/or degradation of oprozomib. In a preferred embodiment, the dosage form increases the absorption of oprozomib in the duodenum and jejunum, leaving less of the drug to be present in the ileum and colon, which can cause tolerability issues. The present dosage form can increase the GI tolerability of oprozomib, which can increase the likelihood of patient compliance with the dosage regimen, which can increase the likelihood of patient compliance with the dosage regimen.

In certain embodiments, a single dose of the dosage form comprising about 60 mg of oprozomib to a dog produces a peak plasma concentration (Cₘₐₓ) of oprozomib of 3.81 ng/mL (having a standard deviation of 2.28).

In certain embodiments, the administration of a single dose of the dosage form (about 60 mg of oprozomib) to a dog produces an area under the concentration time curve to the last time point (AUC) of oprozomib of 15.6 ng*hr/mL (having a standard deviation of 17.4).

In some embodiments, the dosage forms disclosed herein (25 mg oprozomib dose, 50 mg oprozomib dose and 100 mg oprozomib dose) are stable upon actual or simulated storage at 40° C./75% relative humidity for at least 1 month.

Stability studies were carried out on development batches and clinical batches of the gastro-retentive modified release dosage form using one of the following procedures:

(A) Tablets were packaged in 75 cc white HDPE bottles with desiccant and closures and stored at 2° C. to 8° C. Tablets were tested for appearance, assay and impurities and dissolution at pre-determined time points.

(B) Tablets were packaged in 75 cc white HDPE bottles with desiccant and closures and stored at 25° C.±2° C./60% relative humidity (RH)±5% RH. Tablets were tested for appearance, assay and impurities and dissolution at pre-determined time points.

(C) Tablets were packaged in 75 cc white HDPE bottles with desiccant and closures and stored at 40° C.±2° C./75% RH±5% RH. Tablets were tested for appearance, assay and impurities and dissolution at pre-determined time points.

Stability testing results of development batches of the dosage form showed no significant changes in description, assay, and organic impurities after 3 months at 2° C. to 8° C., 25° C./60% RH, and 40° C./75% RH. No significant changes in dissolution profiles were observed at 2° C. to 8° C. and 25° C./60% RH up to 3 months. Minor variations in water content with no obvious trends were observed over the course of 3 months but there was no corresponding change in other attributes.

Results of the clinical batches showed no significant changes in description, assay, organic impurities, dissolution and water content after 1 month at 2° C. to 8° C., 25° C./60% RH, and 40° C./75% RH

In preferred embodiments, when the dosage form is stored in a 75 cc white HDPE bottle with desiccant at 25° C./60% RH for at least 1 month, the dosage form shows less than about 1.0% degradation of oprozomib. In more preferred embodiments, the amount of degradation of oprozomib is less than 0.5%, 0.4%, 0.3%, 0.2%, and in some instances, less than 0.1%.

In particular, impurities PR-059176 (PR-176) and PR-487 were detected and measured.

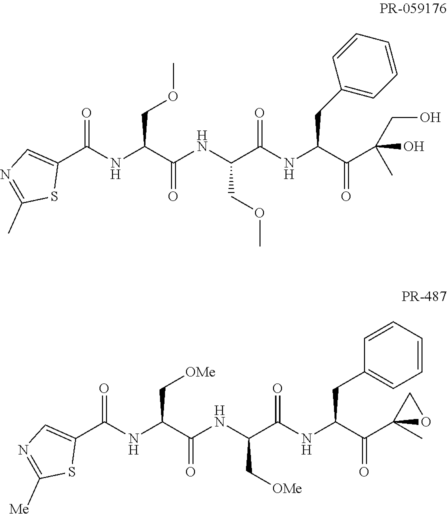 [SMILES: COC[C@H](NC(=O)c1cnc(C)s1)C(=O)N[C@@H](COC)C(=O)N[C@@H](Cc1ccccc1)C(=O)[C@](C)(O)CO ; COC[C@H](NC(=O)c1cnc(C)s1)C(=O)N[C@H](COC)C(=O)N[C@@H](Cc1ccccc1)C(=O)[C@@]1(C)CO1]

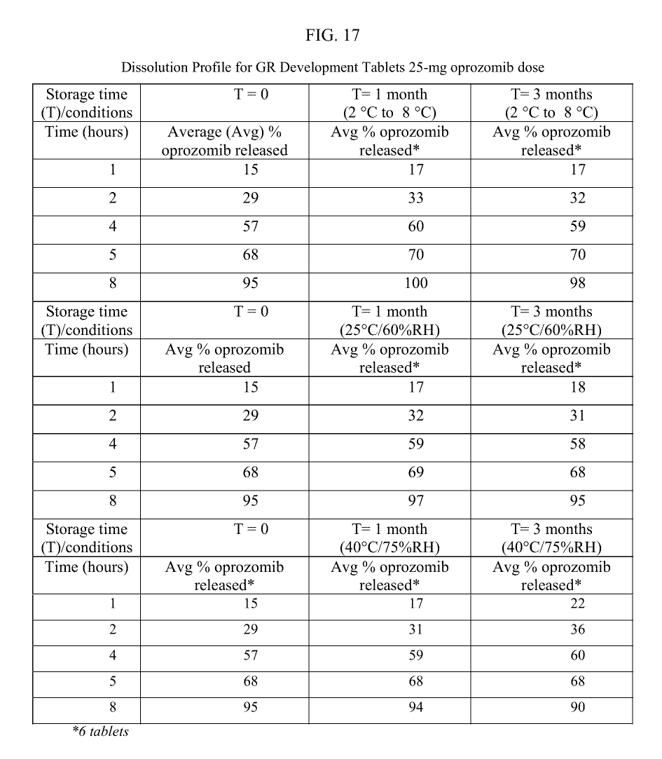

Stability data for the development batches of the 25 mg strength GR tablet described in Table 1 are presented in FIG. 17.

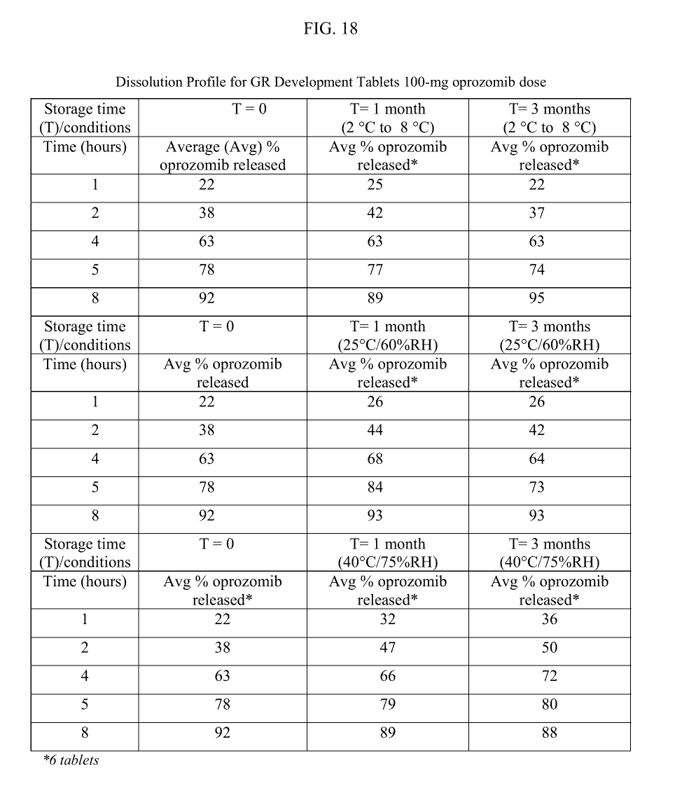

Stability data for the development batches of the 100 mg strength GR tablet described in Table 1 are presented in FIG. 18.

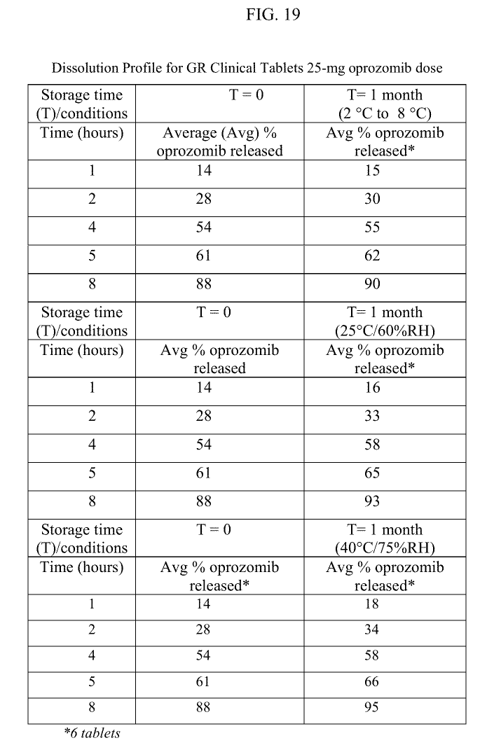

Stability data for the clinical batches of the 25 mg strength GR tablet described in Table 1 are presented in FIG. 19.

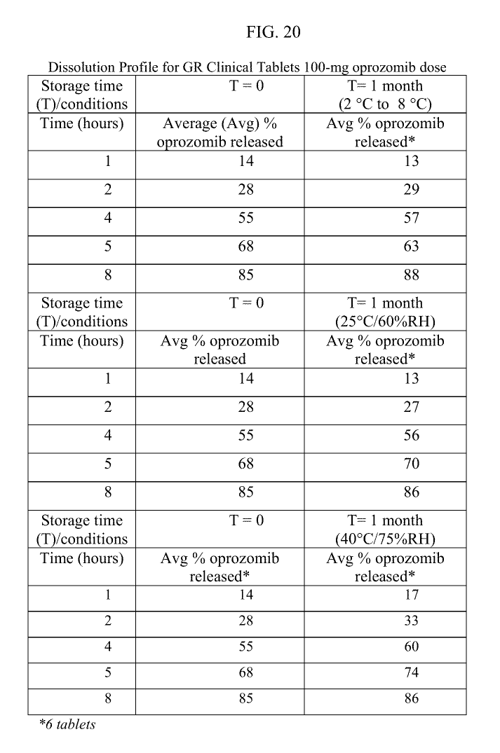

Stability data for the clinical batches of the 100 mg strength GR tablet described in Table 1 are presented in FIG. 20.

Uses of Dosage Forms

Orderly protein degradation is crucial to the maintenance of normal cell functions, and the proteasome is integral to the protein degradation process. The proteasome controls the levels of proteins that are important for cell-cycle progression and apoptosis in normal and malignant cells; for example, cyclins, caspases, BCL2 and NF-κB (Kumatori et al., Proc. Natl. Acad. Sci. USA (1990) 87:7071-7075; Almond et al., Leukemia (2002) 16: 433-443). Thus, it is not surprising that inhibiting proteasome activity can translate into therapies to treat various disease states, such as malignant, non-malignant and autoimmune diseases, depending on the cells involved.

Both in vitro and in vivo models have shown that malignant cells, in general, are susceptible to proteasome inhibition. In fact, proteasome inhibition has already been validated as a therapeutic strategy for the treatment of multiple myeloma. This could be due, in part, to the highly proliferative malignant cell's dependency on the proteasome system to rapidly remove proteins (Rolfe et al., J. Mol. Med. (1997) 75:5-17; Adams, Nature (2004) 4: 349-360). Therefore, certain embodiments of the invention relate to a method of treating a cancer, comprising administering to a subject in need of such treatment an effective amount of a proteasome inhibitor compound disclosed herein. As used herein, the term “cancer” includes, but is not limited to, blood borne and solid tumors. Cancer refers to disease of blood, bone, organs, skin tissue and the vascular system, including, but not limited to, cancers of the bladder, blood, bone, brain, breast, cervix, chest, colon, endometrium, esophagus, eye, head, kidney, liver, lung, lymph nodes, mouth, neck, ovaries, pancreas, prostate, rectum, renal, skin, stomach, testis, throat, and uterus. Specific cancers include, but are not limited to, leukemia (acute lymphocytic leukemia (ALL), acute myelogenous leukemia (AML), chronic lymphocytic leukemia (CLL), chronic myelogenous leukemia (CIVIL), hairy cell leukemia), mature B cell neoplasms (small lymphocytic lymphoma, B cell prolymphocytic leukemia, lymphoplasmacytic lymphoma (such as Waldenström's macroglobulinemia or indolent lymphoma), splenic marginal zone lymphoma, plasma cell myeloma, plasma cell leukemia, plasmacytoma, monoclonal immunoglobulin deposition diseases, heavy chain diseases, extranodal marginal zone B cell lymphoma (MALT lymphoma), nodal marginal zone B cell lymphoma (NMZL), a gastrointestinal tumor (e.g., a gastrointestinal stromal tumor (GIST)), follicular lymphoma, mantle cell lymphoma/leukemia, diffuse B cell lymphoma, mediastinal (thymic) large B cell lymphoma, intravascular large B cell lymphoma, primary effusion lymphoma and Burkitt lymphoma/leukemia), mature T cell and natural killer (NK) cell neoplasms (T cell prolymphocytic leukemia, T cell large granular lymphocytic leukemia, aggressive NK cell leukemia, adult T cell leukemia/lymphoma, extranodal NK/T cell lymphoma, enteropathy-type T cell lymphoma, hepatosplenic T cell lymphoma, blastic NK cell lymphoma, mycosis fungoides (Sezary syndrome), primary cutaneous anaplastic large cell lymphoma, lymphomatoid papulosis, angioimmunoblastic T cell lymphoma, unspecified peripheral T cell lymphoma and anaplastic large cell lymphoma), Hodgkin's lymphoma (nodular sclerosis, mixed celluarity, lymphocyte-rich, lymphocyte depleted or not depleted, nodular lymphocyte-predominant), myeloma (multiple myeloma, indolent myeloma, smoldering myeloma), chronic myeloproliferative disease, myelodysplastic/myeloproliferative disease, myelodysplastic syndromes, immunodeficiency-associated lymphoproliferative disorders, histiocytic and dendritic cell neoplasms, mastocytosis, chondrosarcoma, Ewing sarcoma, fibrosarcoma, malignant giant cell tumor, myeloma bone disease, osteosarcoma, breast cancer (hormone dependent, hormone independent), gynecological cancers (cervical, endometrial, fallopian tube, gestational trophoblastic disease, ovarian, peritoneal, uterine, vaginal and vulvar), basal cell carcinoma (BCC), squamous cell carcinoma (SCC), malignant melanoma, dermatofibrosarcoma protuberans, Merkel cell carcinoma, Kaposi's sarcoma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, malignant mesothelioma (peritoneal mesothelioma, pericardial mesothelioma, pleural mesothelioma), gastro-entero-pancreatic or gastroenteropancreatic neuroendocrine tumor (GEP-NET), carcinoid, pancreatic endocrine tumor (PET), colorectal adenocarcinoma, colorectal carcinoma, aggressive neuroendocrine tumor, leiomyosarcoma, mucinous adenocarcinoma, Signet Ring cell adenocarcinoma, hepatocellular carcinoma, cholangiocarcinoma, hepatoblastoma, hemangioma, hepatic adenoma, focal nodular hyperplasia (nodular regenerative hyperplasia, hamartoma), non-small cell lung carcinoma (NSCLC) (squamous cell lung carcinoma, adenocarcinoma, large cell lung carcinoma), small cell lung carcinoma, thyroid carcinoma, prostate cancer (hormone refractory, androgen independent, androgen dependent, hormone-insensitive), renal cell carcinoma, and soft tissue sarcomas (fibrosarcoma, malignant fibrous hystiocytoma, dermatofibrosarcoma, liposarcoma, rhabdomyosarcoma leiomyosarcoma, hemangiosarcoma, synovial sarcoma, malignant peripheral nerve sheath tumor/neurofibrosarcoma, extraskeletal osteosarcoma).

Many tumors of the haematopoietic and lymphoid tissues are characterized by an increase in cell proliferation, or a particular type of cell. The chronic myeloproliferative diseases (CMPDs) are clonal haematopoietic stem cell disorders characterized by proliferation in the bone marrow of one or more of the myeloid lineages, resulting in increased numbers of granulocytes, red blood cells and/or platelets in the peripheral blood. As such, the use of proteasome inhibitors for the treatment of such diseases is attractive and being examined (Cilloni et al., Haematologica (2007) 92: 1124-1229). CMPD can include chronic myelogenous leukaemia, chronic neutrophilic leukaemia, chronic eosinophilic leukaemia, polycythaemia vera, chronic idiopathic myelofibrosis, essential thrombocythaemia and unclassifiable chronic myeloproliferative disease. An aspect of the invention is the method of treating CMPD comprising administering to a subject in need of such treatment an effective amount of a proteasome inhibitor compound disclosed herein.

Myelodysplastic/myeloproliferative diseases, such as chronic myelomonocytic leukaemia, atypical chronic myeloid leukemia, juvenile myelomonocytic leukaemia and unclassifiable myelodysplastic/myeloproliferative disease, are characterized by hypercellularity of the bone marrow due to proliferation in one or more of the myeloid lineages. Inhibiting the proteasome with a compound or dosage form as described herein can serve to treat these myelodysplatic/myeloproliferative diseases by providing a subject in need of such treatment an effective amount of the compound or dosage form.

Myelodysplastic syndromes (MDS) refer to a group of hematopoietic stem cell disorders characterized by dysplasia and ineffective haematopoiesis in one or more of the major myeloid cell lines. Targeting NF-κB with a proteasome inhibitor in these hematologic malignancies induces apoptosis, thereby killing the malignant cell (Braun et al. Cell Death and Differentiation (2006) 13:748-758). A further embodiment of the invention is a method to treat MDS comprising administering to a subject in need of such treatment an effective amount of a compound disclosed herein. MDS includes refractory anemia, refractory anemia with ringed sideroblasts, refractory cytopenia with multilineage dysplasia, refractory anemia with excess blasts, unclassifiable myelodysplastic syndrome and myelodysplastic syndrome associated with isolated del(5q) chromosome abnormality.

Mastocytosis is a proliferation of mast cells and their subsequent accumulation in one or more organ systems. Mastocytosis includes, but is not limited to, cutaneous mastocytosis, indolent systemic mastocytosis (ISM), systemic mastocytosis with associated clonal haematological non-mast-cell-lineage disease (SM-AHNMD), aggressive systemic mastocytosis (ASM), mast cell leukemia (MCL), mast cell sarcoma (MCS) and extracutaneous mastocytoma. Another embodiment of the invention is a method to treat mastocytosis, comprising administering an effective amount of a compound or dosage form disclosed herein to a subject diagnosed with mastocytosis.

The proteasome regulates NF-κB, which in turn regulates genes involved in the immune and inflammatory response. For example, NF-κB is required for the expression of the immunoglobulin light chain κ gene, the IL-2 receptor α-chain gene, the class I major histocompatibility complex gene, and a number of cytokine genes encoding, for example, IL-2, IL-6, granulocyte colony-stimulating factor, and IFN-β (Palombella et al., Cell (1994) 78:773-785). Thus, in certain embodiments, the invention relates to methods of affecting the level of expression of IL-2, MHC-I, IL-6, TNFα, IFN-β or any of the other previously-mentioned proteins, each method comprising administering to a subject an effective amount of a proteasome inhibitor compound or dosage form disclosed herein. In certain embodiments, the invention includes a method of treating an autoimmune disease in a mammal comprising administering a therapeutically effective amount of a compound or dosage form described herein. An “autoimmune disease” herein is a disease or disorder arising from and directed against an individual's own tissues. Examples of autoimmune diseases or disorders include, but are not limited to, inflammatory responses such as inflammatory skin diseases including psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g., Type I diabetes mellitus or insulin dependent diabetes mellitus); multiple sclerosis; Reynaud's syndrome; autoimmune thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Beheet disease; giant cell arteritis; immune complex nephritis; IgA nephropathy; IgM polyneuropathies; immune thrombocytopenic purpura (ITP) or autoimmune thrombocytopenia.

The immune system screens for autologous cells that are virally infected, have undergone oncogenic transformation, or present unfamiliar peptides on their surface. Intracellular proteolysis generates small peptides for presentation to T-lymphocytes to induce MEW class I-mediated immune responses. Thus, in certain embodiments, the invention relates to a method of using the compound as an immunomodulatory agent for inhibiting or altering antigen presentation in a cell, comprising exposing the cell (or administering to a subject) to a compound described herein. Specific embodiments include a method of treating graft or transplant-related diseases, such as graft-versus-host disease or host versus-graft disease in a mammal, comprising administering a therapeutically effective amount of a compound described herein. The term “graft” as used herein refers to biological material derived from a donor for transplantation into a recipient. Grafts include such diverse material as, for example, isolated cells such as islet cells; tissue such as the amniotic membrane of a newborn, bone marrow, hematopoietic precursor cells, and ocular tissue, such as corneal tissue; and organs such as skin, heart, liver, spleen, pancreas, thyroid lobe, lung, kidney, tubular organs (e.g., intestine, blood vessels, or esophagus). The tubular organs can be used to replace damaged portions of esophagus, blood vessels, or bile duct. The skin grafts can be used not only for burns, but also as a dressing to damaged intestine or to close certain defects such as diaphragmatic hernia. The graft is derived from any mammalian source, including human, whether from cadavers or living donors. In some cases, the donor and recipient is the same mammal. Preferably the graft is bone marrow or an organ such as heart and the donor of the graft and the host are matched for HLA class II antigens.

Histiocytic and dendritic cell neoplasms are derived from phagocytes and accessory cells, which have major roles in the processing and presentation of antigens to lymphocytes. Depleting the proteasome content in dendritic cells has been shown to alter their antigen-induced responses (Chapatte et al. Cancer Res. (2006) 66:5461-5468). Thus, another embodiment of the invention comprises administering an effective amount of a compound or dosage form disclosed herein to a subject with histiocytic or dendritic cell neoplasm. Histiocytic and dendritic cell neoplasms include histiocytic sarcoma, Langerhans cell histiocytosis, Langerhans cell sarcoma, interdigitating dendritic cell sarcoma/tumor, follicular dendritic cell sarcoma/tumor and non-specified dendritic cell sarcoma.

Inhibition of the proteasome has been shown to be beneficial to treat diseases whereby a cell type is proliferating and immune disorders; thus, an embodiment of the invention includes the treatment of lymphoproliferative diseases (LPD) associated with primary immune disorders (PID) comprising administering an effective amount of the disclosed compound to a subject in need thereof. The most common clinical settings of immunodeficiency associated with an increased incidence of lymphoproliferative disorders, including B-cell and T-cell neoplasms and lymphomas, are primary immunodeficiency syndromes and other primary immune disorders, infection with the human immunodeficiency virus (HIV), iatrogenic immunosuppression in patients who have received solid organ or bone marrow allografts, and iatrogenic immunosuppression associated with methotrexate treatment. Other PIDs commonly associated with LPDs, but not limited to, are ataxia telangiectasia (AT), Wiskott-Aldrich syndrome (WAS), common variable immunodeficiency (CVID), severe combined immunodeficiency (SCID), X-linked lymphoproliferative disorder (XLP), Nijmegen breakage syndrome (NBS), hyper-IgM syndrome, and autoimmune lymphoproliferative syndrome (ALPS).

Additional embodiments of the invention relate to methods for affecting the proteasome-dependent regulation of oncoproteins and methods of treating or inhibiting cancer growth, each method comprising exposing a cell (in vivo, e.g., in a subject, or in vitro) to the proteasome inhibitor dosage form disclosed herein. HPV-16 and HPV-18-derived E6 proteins stimulate ATP- and ubiquitin-dependent conjugation and degradation of p53 in crude reticulocyte lysates. The recessive oncogene p53 has been shown to accumulate at the nonpermissive temperature in a cell line with a mutated thermolabile E1. Elevated levels of p53 may lead to apoptosis. Examples of proto-oncoproteins degraded by the ubiquitin system include c-Mos, c-Fos, and c-Jun. In certain embodiments, the invention relates to a method for treating p53-related apoptosis, comprising administering to a subject an effective amount of a proteasome inhibitor dosage form disclosed herein.

Another aspect of the invention relates to the use of proteasome inhibitor dosage forms disclosed herein for the treatment of neurodegenerative diseases and conditions, including, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis).

Alzheimer's disease is characterized by extracellular deposits of β-amyloid protein (β-AP) in senile plaques and cerebral vessels. β-AP is a peptide fragment of 39 to 42 amino acids derived from an amyloid protein precursor (APP). At least three isoforms of APP are known (695, 751, and 770 amino acids). Alternative splicing of mRNA generates the isoforms; normal processing affects a portion of the β-AP sequence, thereby preventing the generation of β-AP. It is believed that abnormal protein processing by the proteasome contributes to the abundance of β-AP in the Alzheimer brain. The APP-processing enzyme in rats contains about ten different subunits (22 kDa-32 kDa). The 25 kDa subunit has an N-terminal sequence of X-Gln-Asn-Pro-Met-X-Thr-Gly-Thr-Ser, which is identical to the β-subunit of human macropain (Kojima, S. et al., Fed. Eur. Biochem. Soc., (1992) 304:57-60). The APP-processing enzyme cleaves at the Gln¹⁵-Lys¹⁶ bond; in the presence of calcium ion, the enzyme also cleaves at the Met⁻¹-Asp¹ bond and the Asp¹-Ala² bond to release the extracellular domain of β-AP.

One aspect of the invention, therefore, relates to a method of treating Alzheimer's disease, comprising administering to a subject an effective amount of a proteasome inhibitor compound or dosage form disclosed herein. Such treatment includes reducing the rate of β-AP processing, reducing the rate of β-AP plaque formation, reducing the rate of β-AP generation, and reducing the clinical signs of Alzheimer's disease.

In some embodiments, a proteasome inhibitor compound or dosage form disclosed herein can be useful for treating amyloidosis. Accordingly, provided herein is a method for treating amyloidosis is a subject, comprising administering to a subject an effective amount of a proteasome inhibitor compound or dosage form disclosed herein.

Fibrosis is the excessive and persistent formation of fibrous connective tissue resulting from the hyperproliferative growth of fibroblasts and is associated with activation of the TGF-β signaling pathway. Fibrosis involves extensive deposition of extracellular matrix and can occur within virtually any tissue or across several different tissues. Normally, the level of intracellular signaling protein (Smad) that activates transcription of target genes upon TGF-β stimulation is regulated by proteasome activity (Xu et al., 2000). However, accelerated degradation of the TGF-signaling components has been observed in fibrotic conditions, such as cystic fibrosis, injection fibrosis, endomyocardial fibrosis, idiopathic pulmonary fibrosis, myelofibrosis, retroperitoneal fibrosis, progressive massive fibrosis, nephrogenic systemic fibrosis. Other conditions that are often associated with fibrosis include cirrhosis, diffuse parenchymal lung disease, post-vasectomy pain syndrome, tuberculosis, sickle-cell anemia and rheumatoid arthritis. An embodiment of the invention is the method of treating a fibrotic or fibrotic-associated condition comprising administering an effective amount of the dosage form described herein to a subject in need of such treatment.

The treatment of burn victims is often hampered by fibrosis. Thus, in certain embodiments, the invention relates to the topical or systemic administration of a subject inhibitor to treat burns. Wound closure following surgery is often associated with disfiguring scars, which may be prevented by inhibition of fibrosis. Thus, in certain embodiments, the invention relates to a method for the prevention or reduction of scarring.

Overproduction of lipopolysaccharide (LPS)-induced cytokines such as TNFα is considered to be central to the processes associated with septic shock. Furthermore, it is generally accepted that the first step in the activation of cells by LPS is the binding of LPS to specific membrane receptors. The α- and β-subunits of the 20S proteasome complex have been identified as LPS-binding proteins, suggesting that the LPS-induced signal transduction may be an important therapeutic target in the treatment or prevention of sepsis (Qureshi, N. et al., J. Immun. (2003) 171: 1515-1525). Therefore, in certain embodiments, the proteasome inhibitor dosage form may be used for the inhibition of TNFα to prevent and/or treat septic shock.

Ischemia and reperfusion injury results in hypoxia, a condition in which there is a deficiency of oxygen reaching the tissues of the body. This condition causes increased degradation of Iκ-Bα, thereby resulting in the activation of NF-κB (Koong et al., 1994). It has been demonstrated that the severity of injury resulting in hypoxia can be reduced with the administration of a proteasome inhibitor (Gao et al., 2000; Bao et al., 2001; Pye et al., 2003). Therefore, certain embodiments of the invention relate to a method of treating an ischemic condition or reperfusion injury comprising administering to a subject in need of such treatment an effective amount of the proteasome inhibitor compound disclosed herein. Examples of such conditions or injuries include, but are not limited to, acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

NF-κB also binds specifically to the HIV-enhancer/promoter. When compared to the Nef of mac239, the HIV regulatory protein Nef of pbj14 differs by two amino acids in the region which controls protein kinase binding. It is believed that the protein kinase signals the phosphorylation of IκB, triggering IκB degradation through the ubiquitin-proteasome pathway. After degradation, NF-κB is released into the nucleus, thus enhancing the transcription of HIV (Cohen, J., Science, (1995) 267:960). In certain embodiments, the invention relates to a method for inhibiting or reducing HIV infection in a subject, or a method for decreasing the level of viral gene expression, each method comprising administering to the subject an effective amount of a proteasome inhibitor compound or dosage form disclosed herein.

Viral infections contribute to the pathology of many diseases. Heart conditions such as ongoing myocarditis and dilated cardiomyopathy have been linked to the coxsackievirus B3. In a comparative whole-genome microarray analyses of infected mouse hearts, specific proteasome subunits were uniformly up-regulated in hearts of mice which developed chronic myocarditis (Szalay et al, Am J Pathol 168:1542-52, 2006). Some viruses utilize the ubiquitin-proteasome system in the viral entry step where the virus is released from the endosome into the cytosol. The mouse hepatitis virus (MHV) belongs to the Coronaviridae family, which also includes the severe acute respiratory syndrome (SARS) coronavirus. Yu and Lai (J Virol 79:644-648, 2005) demonstrated that treatment of cells infected with MHV with a proteasome inhibitor resulted in a decrease in viral replication, correlating with reduced viral titer as compared to that of untreated cells. The human hepatitis B virus (HBV), a member of the Hepadnaviridae virus family, likewise requires virally encoded envelop proteins to propagate. Inhibiting the proteasome degradation pathway causes a significant reduction in the amount of secreted envelope proteins (Simsek et al, J Virol 79:12914-12920, 2005). In addition to HBV, other hepatitis viruses (A, C, D and E) may also utilize the ubiquitin-proteasome degradation pathway for secretion, morphogenesis and pathogenesis. Accordingly, in certain embodiments, the invention relates to a method for treating viral infection, such as SARS or hepatitis A, B, C, D and E, comprising contacting a cell with (or administering to a subject) an effective amount of a compound or dosage form disclosed herein.

In certain embodiments, the disclosed dosage forms may be useful for the treatment of a parasitic infection, such as infections caused by protozoan parasites. The proteasome of these parasites is considered to be involved primarily in cell differentiation and replication activities (Paugam et al., Trends Parasitol. 2003, 19(2): 55-59). Furthermore, entamoeba species have been shown to lose encystation capacity when exposed to proteasome inhibitors (Gonzales, et al., Arch. Med. Res. 1997, 28, Spec No: 139-140). In certain such embodiments, the administrative protocols for the proteasome inhibitor dosage forms are useful for the treatment of parasitic infections in humans caused by a protozoan parasite selected from Plasmodium sps. (including P. falciparum, P. vivax, P. malariae, and P. ovale, which cause malaria), Trypanosoma sps. (including T. cruzi, which causes Chagas' disease, and T. brucei which causes African sleeping sickness), Leishmania sps. (including L. amazonesis, L. donovani, L. infantum, L. mexicana, etc.), Pneumocystis carinii (a protozoan known to cause pneumonia in AIDS and other immunosuppressed patients), Toxoplasma gondii, Entamoeba histolytica, Entamoeba invadens, and Giardia lamblia. In certain embodiments, the disclosed proteasome inhibitor dosage forms are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds that act as proteasome inhibitors in the treatment of parasitic diseases are described in WO 98/10779, which is incorporated herein in its entirety.

In certain embodiments, the proteasome inhibitor dosage forms inhibit proteasome activity in a parasite without recovery in red blood cells and white blood cells. In certain such embodiments, the long half-life of blood cells may provide prolonged protection with regard to therapy against recurring exposures to parasites. In certain embodiments, the proteasome inhibitor dosage forms may provide prolonged protection with regard to chemoprophylaxis against future infection.

Prokaryotes have an equivalent to the eukaryote 20S proteasome particle. Although the subunit dosage form of the prokaryote 20S particle is simpler than that of eukaryotes, it has the ability to hydrolyze peptide bonds in a similar manner. For example, the nucleophilic attack on the peptide bond occurs through the threonine residue on the N-terminus of the β-subunits. Thus, an embodiment of this invention relates to a method of treating prokaryotic infections, comprising administering to a subject an effective amount of a proteasome inhibitor compound or dosage form disclosed herein. Prokaryotic infections may include diseases caused by either mycobacteria (such as tuberculosis, leprosy or Buruli ulcer) or archaebacteria.

It has also been demonstrated that inhibitors that bind to the 20S proteasome stimulate bone formation in bone organ cultures. Furthermore, when such inhibitors have been administered systemically to mice, certain proteasome inhibitors increased bone volume and bone formation rates over 70% (Garrett, I. R. et al., J. Clin. Invest. (2003) 111: 1771-1782), therefore suggesting that the ubiquitin-proteasome machinery regulates osteoblast differentiation and bone formation. Therefore, a disclosed proteasome inhibitor compound or dosage form may be useful in the treatment and/or prevention of diseases associated with bone loss, such as osteoporosis.

Thus, in certain embodiments, the invention relates to a method for treating a disease or condition selected from cancer, autoimmune disease, graft or transplant-related condition, neurodegenerative disease, fibrotic-associated condition, ischemic-related conditions, infection (viral, parasitic or prokaryotic) and diseases associated with bone loss, comprising administering a compound or dosage form as disclosed herein.

In certain embodiments, some exemplary uses for the present invention are compounds that may have GI tolerability issues, such as NVD, and have an absorption window in the upper part of the GI tract. Such compounds may include, but are not limited to the following: metformin, ciprofloxacin, and furosemide.

Also provided herein is a conjoint therapy wherein one or more other therapeutic agents are administered with a peptide proteasome inhibitor or a pharmaceutical composition comprising a peptide proteasome inhibitor. Such conjoint treatment may be achieved by way of the simultaneous, sequential, or separate dosing of the individual components of the treatment.

In certain embodiments, a dosage form provided herein (e.g., pharmaceutical dosage forms that include oprozomib) is conjointly administered with one or more other proteasome inhibitor(s).

In certain embodiments, a dosage form provided herein (e.g., pharmaceutical dosage forms that include oprozomib) is conjointly administered with one or more chemotherapeutics. Suitable chemotherapeutics may include, natural products such as vinca alkaloids (i.e. vinblastine, vincristine, and vinorelbine), taxanes (e.g., docetaxel, paclitaxel, e.g., docetaxel), epidipodophyllotoxins (i.e. etoposide, teniposide), antibiotics (dactinomycin (actinomycin D) daunorubicin, doxorubicin and idarubicin; e.g., doxorubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, ifosphamide, cyclophosphamide and analogs, melphalan, chlorambucil, e.g., melphalan), ethylenimines and methylmelamines (hexaamethylmelaamine and thiotepa), alkyl sulfonates (busulfan), nitrosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine); aromatase inhibitors (anastrozole, exemestane, and letrozole); platinum coordination complexes (cisplatin, carboplatin), procarbazine, hydroxyurea, mitotane, aminoglutethimide; DNA binding/Cytotoxic agents (e.g., Zalypsis); histone deacetylase (HDAC) inhibitors (e.g., trichostatin, sodium butyrate, apicidan, suberoyl anilide hydroamic acid (SAHA (Vorinostat)), trichostatin A, depsipeptide, apicidin, A-161906, scriptaid, PXD-101, CHAP, butyric acid, depudecin, oxamflatin, phenylbutyrate, valproic acid, MS275 (N-(2-Aminophenyl)-4-[N-(pyridine-3-ylmethoxy-carbonyl)aminomethyl]benzamide), LAQ824/LBH589, CI994, MGCD0103, ACY-1215, Panobinostat); hormones (i.e. estrogen) and hormone agonists such as leutinizing hormone releasing hormone (LHRH) agonists (goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, a pharmaceutical dosage form as provided herein (e.g., pharmaceutical dosage forms that include oprozomib) is conjointly administered with one or more histone deacetylase (HDAC) inhibitors (e.g., trichostatin, sodium butyrate, apicidan, suberoyl anilide hydroamic acid (“SAHA” (Vorinostat)), trichostatin A, depsipeptide, apicidin, A-161906, scriptaid, PXD-101, CHAP, butyric acid, depudecin, oxamflatin, phenylbutyrate, valproic acid, MS275 (N-(2-Aminophenyl)-4-[N-(pyridine-3-ylmethoxy-carbonyl)aminomethyl]benzamide), LAQ824/LBH589, CI994, MGCD0103, ACY-1215, Panobinostat; e.g., SAHA, ACY-1215, Panobinostat).

In certain embodiments, a pharmaceutical dosage form as provided herein (e.g., pharmaceutical dosage forms that include oprozomib) is conjointly administered with one or more nitrogen mustards (mechlorethamine, ifosphamide, cyclophosphamide and analogs, melphalan, chlorambucil, e.g., melphalan).

In certain embodiments, a pharmaceutical dosage form as provided herein (e.g., pharmaceutical dosage forms that include oprozomib) is conjointly administered with one or more DNA binding/Cytotoxic agents (e.g., Zalypsis).

In certain embodiments, a pharmaceutical dosage form as provided herein (e.g., pharmaceutical dosage forms that include oprozomib) is conjointly administered with one or more taxanes (e.g., docetaxel, paclitaxel, e.g., docetaxel).

In certain embodiments, a pharmaceutical dosage form as provided (e.g., pharmaceutical dosage forms that include oprozomib) is conjointly administered with one or more antibiotics (dactinomycin (actinomycin D) daunorubicin, doxorubicin and idarubicin; e.g., doxorubicin).

In some embodiments, a pharmaceutical dosage form as provided herein (e.g., pharmaceutical dosage forms that include oprozomib) is conjointly administered with one or more cytokines. Cytokines include, but are not limited to, Interferon-γ, -α, and -β, Interleukins 1-8, 10 and 12, Granulocyte Monocyte Colony-Stimulating factor (GM-CSF), TNF-α and -β, and TGF-β.

In some embodiments, a pharmaceutical dosage form provided herein (e.g., pharmaceutical dosage forms that include oprozomib) is conjointly administered with one or more steroids. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof (e.g., hydrocortisone, dexamethasone, methylprednisolone and prednisolone; e.g., dexamethasone).

The invention will be further described in the following examples. All reagents are commercially available unless otherwise noted. The Examples section provides more specific methods for preparing the dosage forms described herein. It should be understood that these examples are for illustrative purposes only and are not to be construed as limiting this invention in any manner.

### EXAMPLES

### Example 1

Preparation of Oprozomib (Compound 1 in the Example Below)

The following synthesis of Oprozomib has been published in U.S. Pat. No. 9,295,708, hereby incorporated by reference in its entirety).

Synthesis of Compound 1

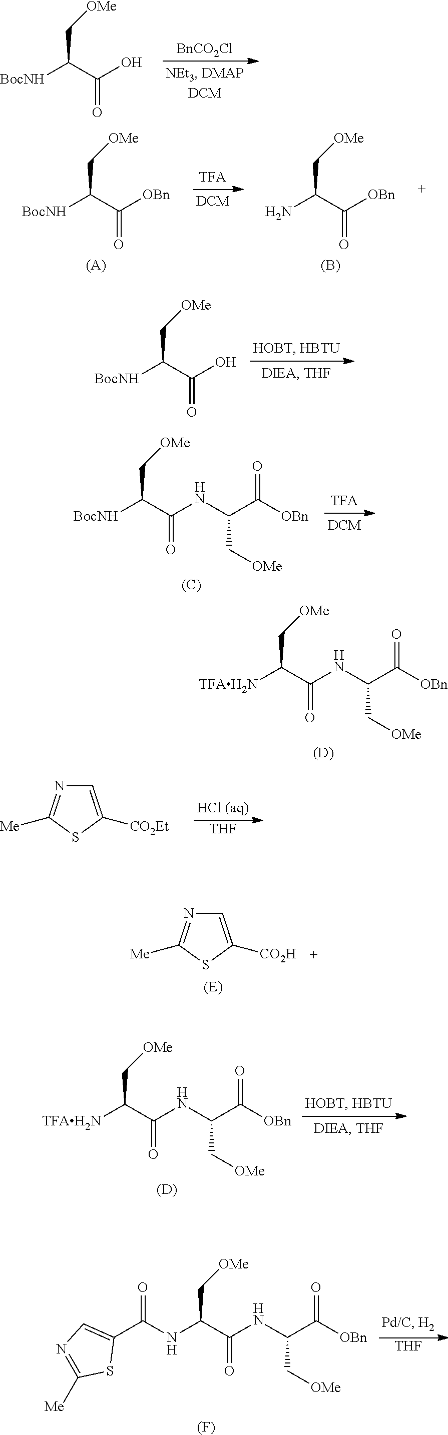

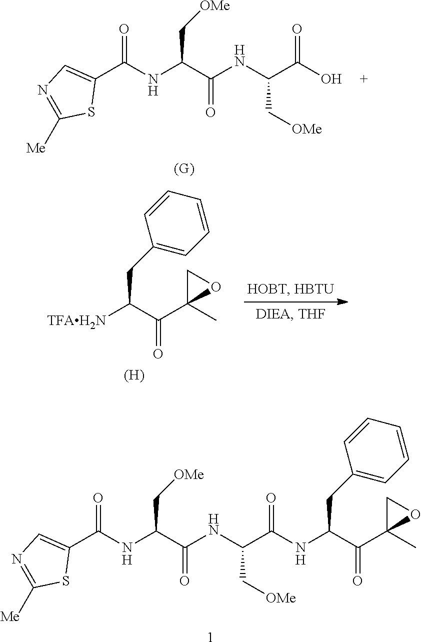

Synthesis of (A)

To a 0° C. solution of N-Boc serine(methyl ether) (43.8 g, 200 mmol), triethylamine (26.5 g, 260 mmol) and 4-(dimethylamino)pyridine in dichloromethane (1.2 L) was added a solution of benzyl chloroformate (41 g, 240 mmol) in dichloromethane (250 mL) over 30 minutes. The resulting mixture was stirred at the same temperature for another 3 hours. Saturated aqueous sodium bicarbonate (200 mL) was added and organic layer was separated, the residual mixture was extracted with dichloromethane (2×400 mL). The combined organic layers were washed with saturated aqueous sodium bicarbonate (200 mL) and brine (200 mL), dried over sodium sulfate and filtered through Celite-545. The solvents were removed under reduced pressure and residue was purified by flash chromatography (silica gel, hexane and ethyl acetate). Compound (A) (54 g) was isolated and characterized by LC/MS (LRMS(MH) m/z: 310.16).

Synthesis of (B)

To a 0° C. solution of Compound (A) (54 g) in dichloromethane (200 mL) was added trifluoroacetic acid (200 mL) over 10 minutes, and the resulting mixture was stirred at the same temperature for another 3 hours. The solvents were removed under reduced pressure and the residue was placed under high vacuum overnight giving the TFA salt of Compound (B), which was characterized by LC/MS (LRMS (MH) m/z: 210.11).

Synthesis of (C)

To a 0° C. solution of Compound (B) (43.8 g, 200 mmol), N-Boc serine(methyl ether) (36.7 g, 167 mmol), HOBT (27 g, 200 mmol) and HBTU (71.4 g, 200 mmol) in tetrahydrofuran (1.2 L) was added a solution of N,N-diethylisopropylamine (75 g, 600 mmol) in tetrahydrofuran (250 mL) over 10 minutes, and the pH of the resulting mixture was ˜8. The mixture was stirred at room temperature for another 5 hours. Most of the solvent were removed under reduced pressure at room temperature and diluted with saturated aqueous sodium bicarbonate (400 mL). Then it was extracted with ethyl acetate (3×400 mL), washed with sodium bicarbonate (100 mL) and brine (100 mL). The combined organic layers were dried over sodium sulfate and filtered through Celite-545. The solvents were removed under reduced pressure and residue was purified by flash chromatography (silica gel, hexane and ethyl acetate). Compound (C) (65 g) was isolated and characterized by LC/MS (LRMS (MH) m/z: 411.21).

Synthesis of (D)

To a 0° C. solution of Compound (C) (18 g) in dichloromethane (100 mL) was added trifluoroacetic acid (80 mL) over 5 minutes, and the resulting mixture was stirred at the same temperature for another 3 hours. The solvents were removed under reduced pressure and the residue was placed under high vacuum overnight giving the TFA salt of intermediate (D), which was characterized by LC/MS (LRMS (MH) m/z: 311.15).

Synthesis of (E)

To a 0° C. solution of ethyl 2-methyl-thiazole-5-carboxylate (15 g, 88 mmol) in tetrahydrofuran (50 mL) was added aqueous sodium hydroxide solution (5 N, 50 mL) over 10 minutes, and the resulting solution was stirred at room temperature for another 2 hours. It was then acidified with hydrochloric acid (2 N) to pH=1 and extracted with tetrahydrofuran (3×100 mL). The combined organic layers were washed with brine (30 mL) and dried over sodium sulfate. Most of the solvents were removed under reduced pressure and the residue was lyophilized to afford Compound (E) (14 g).

Synthesis of (F)

To a 0° C. solution of Compound (D) (41 mmol) and 2-methyl-thiazole-5-carboxylic acid (E) (6.0 g, 42 mmol), HOBT (7.9 g, 50 mmol) and HBTU (18.0 g, 50 mmol) in tetrahydrofuran (800 mL) was added a solution of N,N-diethylisopropylamine (˜50 g) in tetrahydrofuran (200 mL) over 5 minutes until its pH reached approximately 8.5. The resulting mixture was stirred at same temperature overnight. It was then quenched with saturated aqueous sodium bicarbonate solution (200 mL), and most of the solvents were removed under reduced pressure. The residual mixture was extracted with ethyl acetate (3×400 mL). The combined organic layers were washed with saturated aqueous sodium bicarbonate (200 mL) and brine (100 mL), dried over sodium sulfate and filtered through Celite-545. The solvents were removed under reduced pressure and residue was purified by flash chromatography (silica gel, ethyl acetate with 2% methanol). Compound (F) (17.1 g) was isolated and characterized by LC/MS (LRMS (MH) m/z: 436.15).

Synthesis of (G)

To a solution of Compound (F) (17.1 g, 95 mmol) in methanol (300 mL) was added 10% Pd/C (3 g). The resulting mixture was allowed to stir under 1 atmosphere of hydrogen for 48 hours. The mixture was filtered through Celite 545 and the filter cake was washed with methanol (˜200 mL). The organic layers were concentrated under reduced pressure and placed under high vacuum to yield Compound (G), which was characterized by LC/MS (LRMS (MH) m/z: 346.1).

Synthesis of (H)

N-Boc phenylalanine-ketoepoxide (140 mg, 0.46 mmol) was diluted with DCM (2 mL) and cooled to 0° C. To this solution was added trifluoroacetic acid (6 mL). The cooling bath was removed and the reaction stirred for 1 hour at which time TLC showed complete consumption of starting material. The resulting solution was concentrated under reduced pressure and placed under high vacuum to yield TFA salt of Compound (H).

Synthesis of Compound 1

To a 0° C. solution of aforementioned Compounds (H) (131 mg, 0.38 mmol) and (J) (0.46 mmol), HOBT (75 mg, 0.48 mmol) and HBTU (171 mg, 0.48 mmol) in tetrahydrofuran (20 mL) and N,N-dimethylformamide (10 mL) was added N,N-diethylisopropylamine (1 mL) dropwise. The mixture was stirred at the same temperature for another 5 hours. It was then quenched with saturated aqueous sodium bicarbonate solution (20 mL), and most of the solvents were removed under reduced pressure. The residual mixture was then extracted with ethyl acetate (3×40 mL). The combined organic layers were washed with saturated aqueous sodium bicarbonate (20 mL) and brine (10 mL), dried over sodium sulfate and filtered through Celite-545. The solvents were removed under reduced pressure and residue was purified by HPLC (0.02 M aqueous ammonium acetate and acetonitrile (66/34) to afford Compound 1 (92 mg), which was lyophilized and characterized by LC/MS (LRMS (MH) m/z: 533.2).

### Example 2

Amorphous Compound 1 (50 mg) was dissolved in acetonitrile (1 mL), then deionized water (2 mL) was added, and the solution brought to supersaturation by slowly evaporating off 1 mL over about 1-2 weeks. The resulting crystals were filtered, washed with 1 mL 1:2 acetonitrile-water, and dried under vacuum for 12 hours to provide a crystalline polymorph of Compound 1 (25 mg) with a melting point of 148° C. The characteristic DSC curve of the sample is shown in FIG. 3 as recorded on a TA Instruments Differential Scanning Calorimeter 2920 at a heating rate of 10° C./minute.

### Example 3

Amorphous Compound 1 (611 mg) was dissolved in tetrahydrofuran (5 mL), followed by addition of hexanes (5 mL) and the solution was seeded with crystalline polymorph Compound 1 as prepared in Example 2, and the solution brought to supersaturation by slowly evaporating off 5 mL over about 17 hours. The resulting crystals were filtered, washed with 1 mL 1:1 tetrahydrofuran-hexanes, and dried under vacuum for 12 hours to provide a crystalline polymorph of Compound 1 (150 mg) with a melting point of 147° C.

### Example 4

Amorphous Compound 1 (176 mg) was dissolved in tetrahydrofuran (5 mL), then toluene (25 mL) was added. The solution was seeded with crystalline polymorph Compound 1 as prepared in Example 2, and the solution was brought to supersaturation by slowly evaporating off 20 mL over about 2 days. The resulting crystals were filtered, washed with 15 mL toluene, and dried under vacuum for 12 hours to provide a crystalline polymorph of Compound 1 (88 mg) with a melting point of 149° C.

### Example 5

Amorphous Compound 1 (312 mg) was dissolved in toluene (50 mL), heated to about 100° C. to complete dissolution, then hexanes (50 mL) were added and the solution was seeded with crystalline polymorph Compound 1 as prepared in Example 2, and the solution brought to supersaturation by slowly evaporating off 60 mL over about 2 days. The resulting crystals were filtered, washed with 10 mL toluene, and dried under vacuum for 12 hours to provide a crystalline polymorph of Compound 1 (156 mg) with a melting point of 149° C.

### Example 6

Amorphous Compound 1 (1.4 g) was dissolved in toluene (25 mL), heated to about 50° C. to complete dissolution, then brought to supersaturation by cooling to 22° C. and allowing the compound to crystallize for 12 hours. The resulting crystals were filtered, washed with 5 mL hexanes, and dried under vacuum for 12 hours to provide a crystalline polymorph of Compound 1 (0.94 g) with a melting point of 149° C.

### Example 7

Synthesis of Compound 1

Synthesis of (H)

N-Boc phenylalanine-ketoepoxide (1.0 equivalent) was dissolved in DCM (3 L/kg of N-Boc phenylalanine-ketoepoxide) in a 3-neck round bottom flask under inert atmosphere and the solution was cooled in ice bath. Then, TFA (5.0 equivalents) was added at a rate to maintain the internal temperature below 10° C. The reaction mixture was then warmed to approximately 20° C. and stirred for 1 to 3 hours. MTBE (3.6 L/kg of N-Boc phenylalanine-ketoepoxide) was then added to the reaction mixture while maintaining mixture temperature below 25° C. Heptane (26.4 L/kg of N-Boc phenylalanine-ketoepoxide) was then added the reaction was cooled to between −5 and 0° C. for 2 to 3 hours to allow crystallization of Compound (H). The white solid was filtered and rinsed with heptane (3 L/kg of N-Boc phenylalanine-ketoepoxide). The white solid was then under vacuum for 12 hours at 22° C. Yield obtained was 86%, with HPLC purity 99.4%.

Synthesis of Compound 1

Compound (H) (1.2 equivalents), Compound (G) (1.0 equivalent), HBTU (1.2 equivalents), HOBT (1.2 equivalents) and N-methyl pyrrolidinone (8 L/kg of Compound (G)) were added to a dry flask under inert atmosphere, and the mixture was stirred at 23° C. to complete dissolution. The reaction was then cooled to between −5 and 0° C., and diisopropylethylamine (2.1 equivalents) was added over 15 minutes, while maintaining an internal reaction temperature of less than 0° C. The reaction mixture was stirred at 0° C. for 12 hours.

Crude Compound 1 was precipitated by pouring the reaction mixture onto 8% sodium bicarbonate (40 L/kg of Compound (G)) and the suspension of crude Compound 1 was stirred for 12 hours at 20 to 25° C., followed by stirring at 0 to 5° C. for 1 hour. The white solid was filtered and rinsed with water (5 L/kg of Compound (G)). The white solid was then reslurried in water (15 L/kg) for 3 hours at 20 to 25° C., filtered and rinsed with water (5 L/kg of Compound (G)) and isopropyl acetate (2×2 L/kg of Compound (G)). The white solid was dried under vacuum at 45° C. to constant weight. Yield of crude Compound 1 was 65%, with HPLC purity of 97.2%.

Crude Compound 1 was completely dissolved in isopropyl acetate (20 L/kg of crude Compound 1) by stirring and heating at 85° C. The solution was then hot filtered to remove any particulate matter and the solution was re-heated to 85° C. to provide clear solution. The clear solution was allowed to cool at 10° C. per hour to 65° C. before adding seed crystals. The solution was allowed to cool at 10° C. per hour to 20° C., when substantial crystallization of Compound 1 occurred. The suspension was stirred at 20° C. for 6 hours, followed by stirring at 0 to 5° C. for a minimum of 2 hours and filtration and rinsing with isopropyl acetate (1 L/kg of crude Compound 1). The purified Compound 1 was dried under vacuum at 45° C. for a minimum of 24 hours to constant weight. Yield of Compound 1 was 87%, with HPLC purity 97.2%.

### Example 8

Synthesis of Compound 1

Compound (H) (1.1 equivalents), Compound (G) (1.0 equivalent), HBTU (1.5 equivalents), HOBT (1.5 equivalents) and DMF (8 L/kg of Compound (G)) were added to a dry flask under inert atmosphere, and the mixture was stirred at 23° C. to complete dissolution. The reaction was then cooled to between −5 and 0° C., and diisopropylethylamine (2.1 equivalents) was added over 15 minutes, while maintaining an internal reaction temperature of less than 0° C. The reaction mixture was then stirred at 0° C. for 3 hours.

The reaction mixture was quenched by addition of pre-chilled saturated sodium bicarbonate (94 L/kg of Compound (G)), while maintaining internal temperature of less 10° C. The content was then transferred to a separatory funnel. The mixture was extracted with ethyl acetate (24 L/kg of Compound (G)), and the organic layer was washed with saturated sodium bicarbonate (12 L/kg of Compound (G)) and with saturated sodium chloride (12 L/kg of Compound (G).

The organic layer was concentrated under reduced pressure with a bath temperature of less than 30° C. to 15 L/kg of Compound (G), followed by co-distillation with isopropyl acetate (2×24 L/kg of PR-022). Final volume was adjusted to 82 L/kg of Compound (G) with isopropyl acetate before heating to 60° C. to obtain a clear solution. The clear solution mixture was allowed to cool to 50° C. before adding seed crystals. The solution was allowed to cool to 20° C., when substantial crystallization of Compound 1 had occurred. The suspension was stirred at 0° C. for 12 hours before filtration and rinsing with isopropyl acetate (2 L/kg of Compound 1). Compound 1 was dried under vacuum at 20° C. for 12 hours to constant weight. Yield of Compound 1 was 48%, with HPLC purity of 97.4%.

### Example 9

Preparation and Analysis of Oprozomib Tablets

Following is a general procedure followed to prepare tablet granulation and compress tablets.

Oprozomib 25 mg and 100 mg GR modified release tablets are manufactured via dry granulation using a roller compaction process.

Dry Granulation

Step 1. Screen oprozomib and microcrystalline cellulose using a metal sieve.

Step 2. Blend the screened components with PolyOx® W SR 1105 LEO polymer in a suitable blender.

Step 3. Blend pre-screened magnesium stearate with materials from Step 2 in a suitable blender.

Step 4. Roller compact the blend into ribbons and subsequently mill the ribbons in the roller compactor equipped with a mill.

Step 5. Blend pre-screened magnesium stearate with PolyOx® WSR 303 LEO polymer in a suitable blender.

Step 6. Compress granules from Step 4 and blend from Step 5 using a rotary bilayer tablet press. The tablet appearance, weight, hardness, and thickness are monitored throughout the compression process.

Tablet Compression and Coating

Tablets weighing about 600 mg with 25 mg and 100 mg drug loading were compressed with 0.3420″×0.5480″ modified oval tooling respectively using a Carver Press or single station press or a rotary tablet press.
Tablets were compressed at predetermined pressure and evaluated for thickness and hardness.
Tablet characteristics and process parameters are documented.
Tablets prepared are stored at either 2° C. to 8° C. or at room temperature (“RT”) until further processed or used.
Tablets were film coated using a perforated pan coater with Opadry® II 85F18422 coating which is a release coating polymer dosage form marketed by Colorcon®.

### Tablet Characterization

Tablets prepared were characterized for thickness, hardness, friability and dissolution characteristics. The tablet granulation was characterized for compressibility index and particle size distribution.
Thickness was measured using a VWR® Electronic Digital Caliper (VWR, Inc., Radnor, Pa.).
Hardness was measured using a Vankel® VK 200 (Agilent Technologies, Santa Clara, Calif.).
Dissolution was performed with USP Apparatus 3 apparatus with 30 dips per minute with a dissolution medium of 0.01N HCl.
Dissolution samples were analyzed using an Agilent® 1100 HPLC system with auto sampler and DAD detector.

### Example 10

Statistical Analysis

If 3 or more units were tested, relative standard deviation (“RSD”) was calculated. RSD was not calculated for 2 or less sample units.

### Example 11

Dosage Form Release Profiles

Since the tablet dosage forms were manufactured using one of the following: Carver press, single station press, or rotary tablet press, the uniformity of the tablets prepared were monitored by measuring the thickness and weights of all the tablets and hardness on a few of them. The desired tablet thickness was defined to be in the range of about 5.0 millimeters to about 8.0 millimeters as measured by the digital calipers. The tablet can have a thickness of from about 6.00 millimeters to about 7.00 millimeters, e.g., about 6.6 mm; e.g., about 6.9 mm. The active pharmaceutical agent particle size is about 6-13 mm in length in maximum dimension prior to swelling.

Tablets outside the desired thickness range were rejected. The tablet hardness is inversely proportional to the thickness (for the current working range) and the thickness and hardness of the tablets were well correlated. The desired average tablet hardness strength was between about 1.00 to about 50.00 kp, e.g., about 16-20 kp. e.g., about 20 kp.

### Example 12

Stability Study

The stability of oprozomib tablets prepared and stored with desiccant at 2° C. to 8° C. and/or RT for more than 1 month were evaluated for assay and impurities and were found to be acceptable without any anomalous peaks implying stability at 2° C. to 8° C. and/or RT. In some embodiments, the 25 mg and 50 mg dosage forms are stable upon actual or simulated storage at 25° C./60% relative humidity for at least 1 month.

Stability studies were carried out using one of the following procedures:

a. The composition of the GR Development and Clinical Tablets (25 mg and 100 mg) used are described in Table 1.

b. Tablets were packaged in 75 cc white HDPE bottles with closures and desiccant and stored at 5° C.±3° C., 25° C.±2° C./60% relative humidity (RH)±5% RH and 40° C.±2° C./75% RH±5% RH. Tablets were tested for appearance, hardness, assay and impurities and dissolution at pre-determined time points (FIGS. 17-20).

c. In preferred embodiments, when the dosage form is stored in a 75 cc HDPE bottle with closures and desiccant at 5° C.±3° C., for at least 3 months (FIGS. 17 and 18), the dosage form shows less than about 1.0% degradation of oprozomib. In more preferred embodiments, the amount of degradation of oprozomib is less than 0.5%, 0.4%, 0.3%, 0.2%, and in some instances, less than 0.1%.

### Example 13

PK/PD Studies

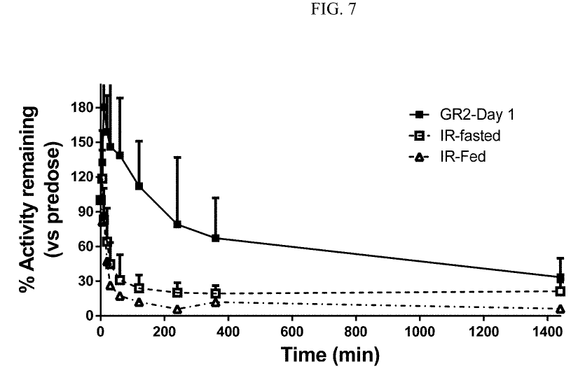

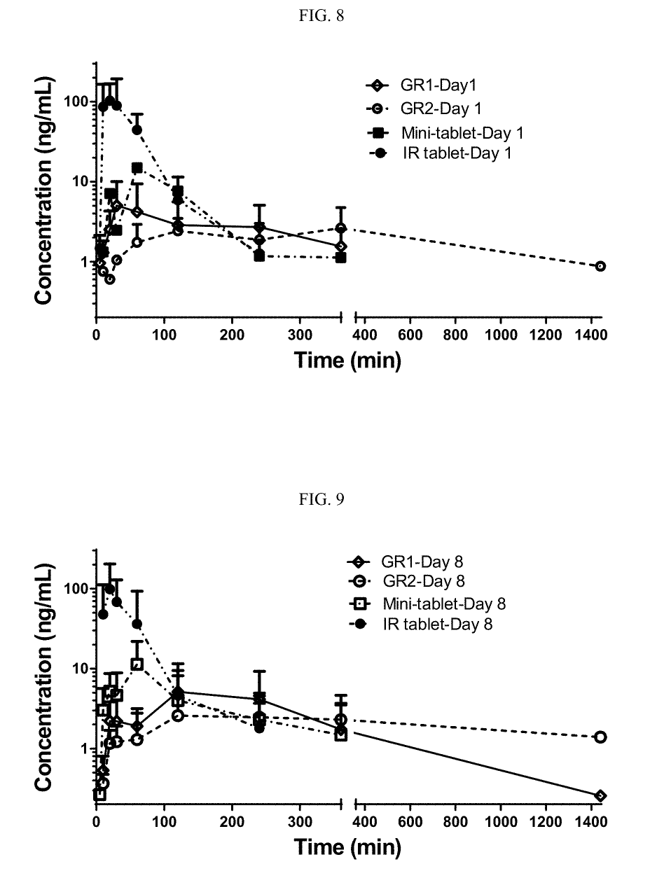

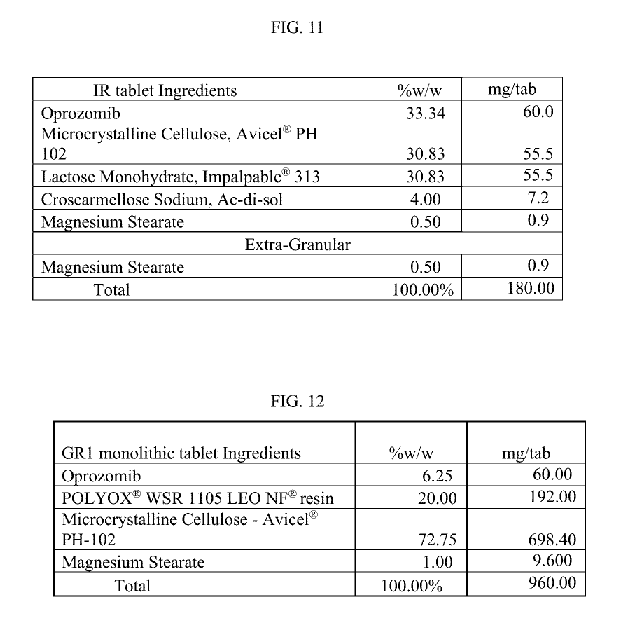

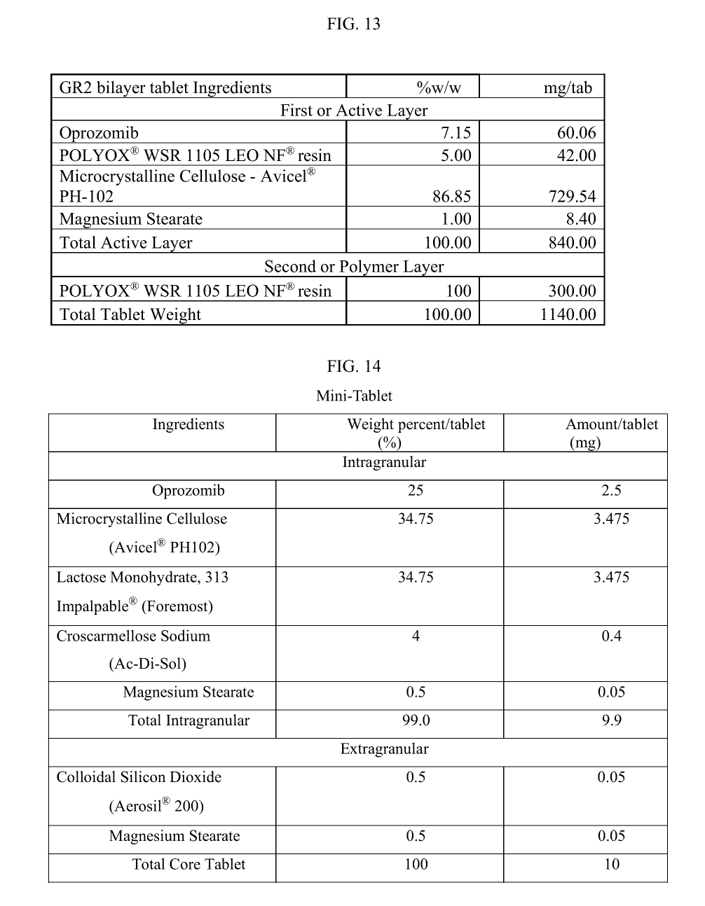

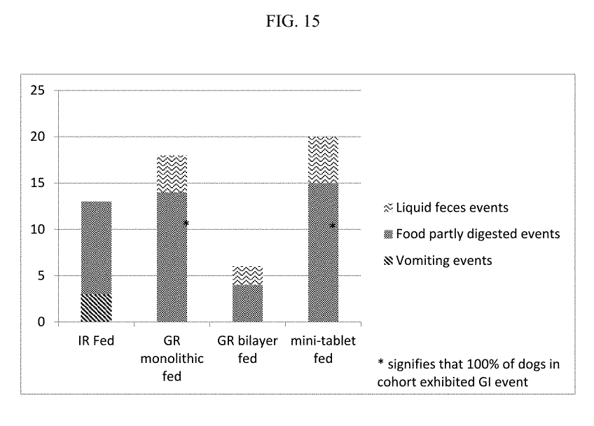

PK/PD (FIGS. 6, 7, 8, 9, 10 and 10(a)) studies were conducted using the following dosage forms: IR tablet (“IR”) (see FIG. 11), GR1 Monolithic tablet (“GR1”) (FIG. 12), the GR2 Bilayer tablet (“GR2”) (FIG. 13), and the mini-tablet (FIG. 14).

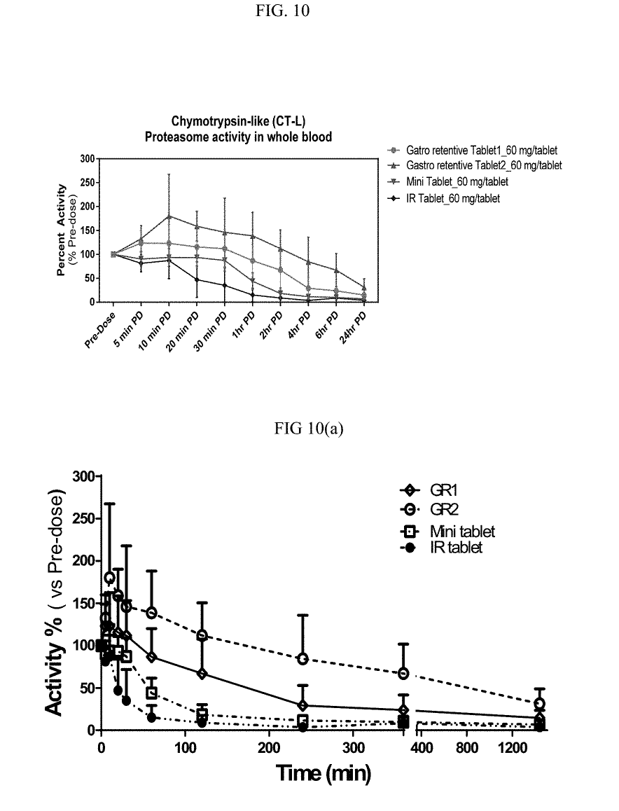

In vivo dog data in the fed state show that oprozomib administration using GR2 dosage forms reduced GI intolerability (such as emesis (vomiting) events (FIG. 15)) relative to IR dosage form while maintaining the PK/PD activity (FIGS. 6, 7, 8, 9, 10 and 10(a)). FIGS. 10 and 10(a) plot the same data but on a slightly different time scale. Female dogs were administered a single dose of 60 mg/kg oprozomib in IR (dogs were fasted unless otherwise noted), GR1 (dogs were fed), GR2 (dogs were fed), mini-tablets (24 of the minitablets were inserted into a capsule; each mini-tablet has a dose of 2.5 mg oprozomib. 24×2.5 mg=60 mg) (dogs were fed), dosage forms. Blood samples were collected from pre-dose to 24 hours post-dose for plasma PK parameter determination and blood PD analyses of proteasome inhibition (FIGS. 6, 7, 8, 9, 10 and 10(a)). GI tolerability were recorded up to 48 hours post-dose (FIG. 15). The GR2 dosage form in the fed state had the lowest maximum concentration and similar exposures as the GR1 tablet (fed state), mini-tablet (fed state), IR tablet (fasted state), but lower exposure than the IR tablet (fed state) (FIGS. 6, 7, 8, 9, 10 and 10(a)). The GR2 dosage forms had a time to peak plasma concentrations of from about 1 to about 8 hours, preferably 4 to 6 hours. Rapid potent inhibition of proteasome activity (100% of pre-dose) was observed for the IR dosage forms. However, the desired long term modified release and exposure of oprozomib was achieved with the GR2 bilayer tablet formulation (FIGS. 6, 7, 8, 9, 10 and 10(a)).

GR2 dosage forms caused less GI intolerability than the IR, GR1 and mini-tablet dosage forms in the fed state (FIG. 15). Following a 60 mg/kg dose, the GR2 dosage form resulted in substantially lower adverse GI events in the fed state while the IR, GR1 and the mini-tablet dosage forms resulted in significantly more adverse GI events. As such, the GR dosage forms described herein can provide a reduced incidence or severity of one or more GI side effects (e.g., NVD, increased salivation).

A single dose of the dosage form GR2 comprising 60 mg of oprozomib to a dog produces peak plasma concentration (Cₘₐₓ) of oprozomib of 66.4 ng/mL (having a standard deviation of 73.3).

The administration of the dosage form GR2 to a dog produces an area under the concentration time curve to the last time point (AUC) of oprozomib of 28.6 ng*hr/mL, having standard deviation of 18.6).

### Example 14. Oprozomib Dosing Regimen

Dogs were dosed once a week on a one time basis, e.g., Day 1 and Day 8. Patients are expected to be administered oprozomib formulated in a tablet form according to either a QDx2 treatment schedule or QDx2 weekly treatment schedule. As used herein, “QDx2” means that patients receive oprozomib tablets once daily on days 1-2 of a 7-day treatment schedule. Patients may be administered oprozomib formulated in a tablet where the patient receives oprozomib on days one through 2 of a seven-day treatment schedule.

The dosage forms of oprozomib may be administered with or without food; however, the GR dosage forms of the present invention are preferably administered after the main meal of the day.

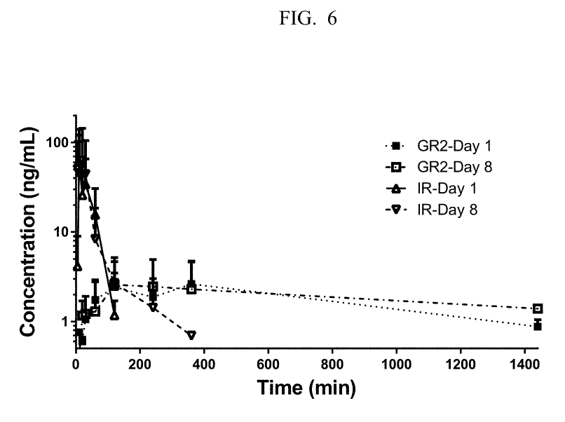

As illustrated in FIG. 1 and exemplified by FIG. 15, the GR modified release bilayer tablet dosage forms described herein can efficiently release oprozomib, e.g., to the stomach and proximal part of the small intestine, and do so over an extended or modified period of time and, in some instances, with improved bioavailability, pharmacokinetic (PK) (FIGS. 6, 8, and 9) and/or pharmacodynamic (PD) (FIGS. 7, 10 and 10(a)) parameters, thereby increasing the likelihood that oprozomib will be absorbed by the duodenum and jejunum prior to excretion and/or degradation of oprozomib. In a preferred embodiment, the GR modified release bilayer tablet dosage form increases the absorption of oprozomib in the duodenum and jejunum, leaving less of the drug to be present in the ileum and colon, which can cause tolerability issues. The present GR modified release bilayer tablet dosage form can increase the GI tolerability of oprozomib (FIG. 15), which can increase the likelihood of patient compliance with the dosage regimen, which can increase the likelihood of patient compliance with the dosage regimen. As such, the GR modified release bilayer tablet dosage forms described herein can provide a reduced incidence or severity of one or more GI side effects (e.g., NVD).

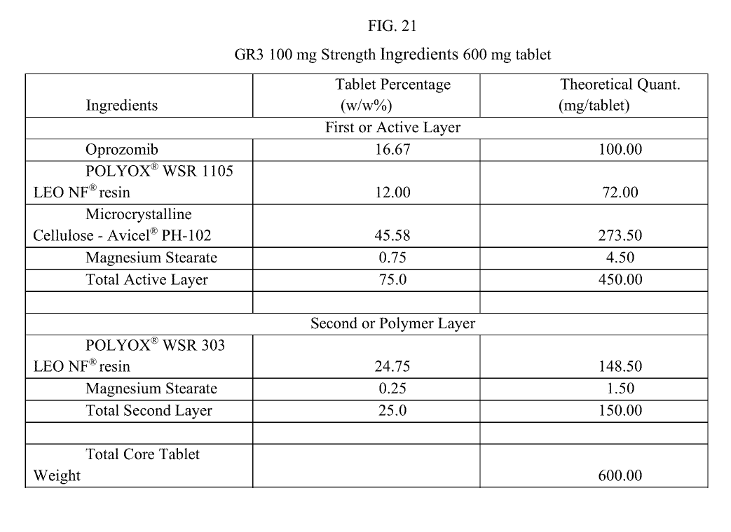

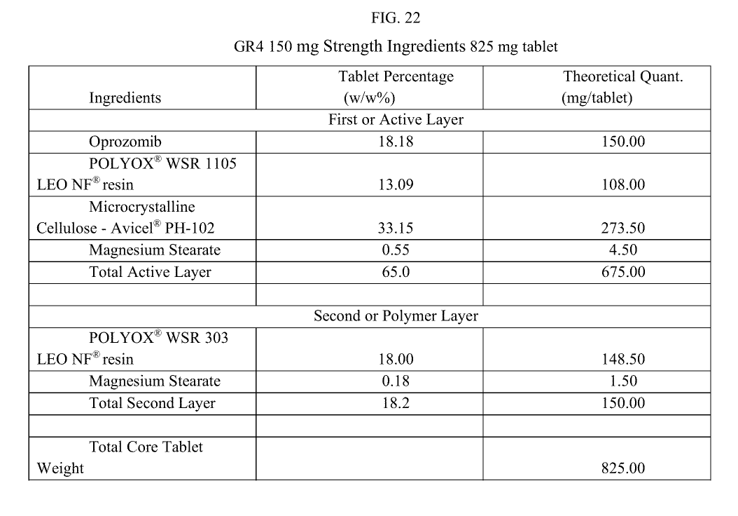

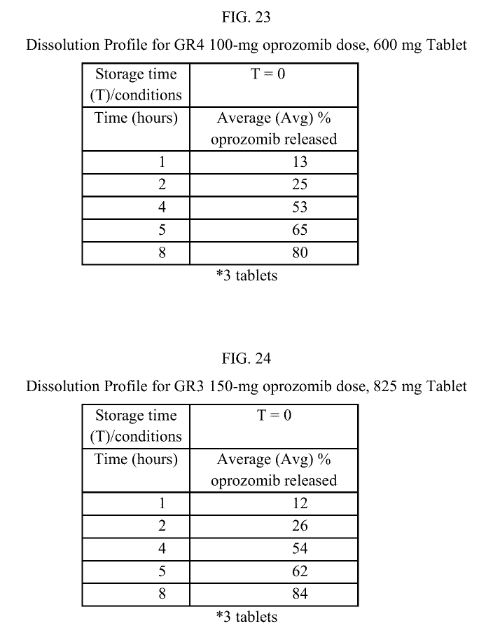

Additional GR modified release bilayer tablet dosage forms described herein (GR3, FIG. 21 and GR4, FIG. 22) can efficiently release oprozomib, e.g., to the stomach and proximal part of the small intestine, and do so over an extended or modified period of time. FIG. 23 illustrates the Dissolution Profile for GR3 modified release Clinical Tablets 100-mg oprozomib dose (600 mg). FIG. 24 illustrates the Dissolution Profile for GR4 modified release Clinical Tablets 150-mg oprozomib dose (825 mg).

### Process Development

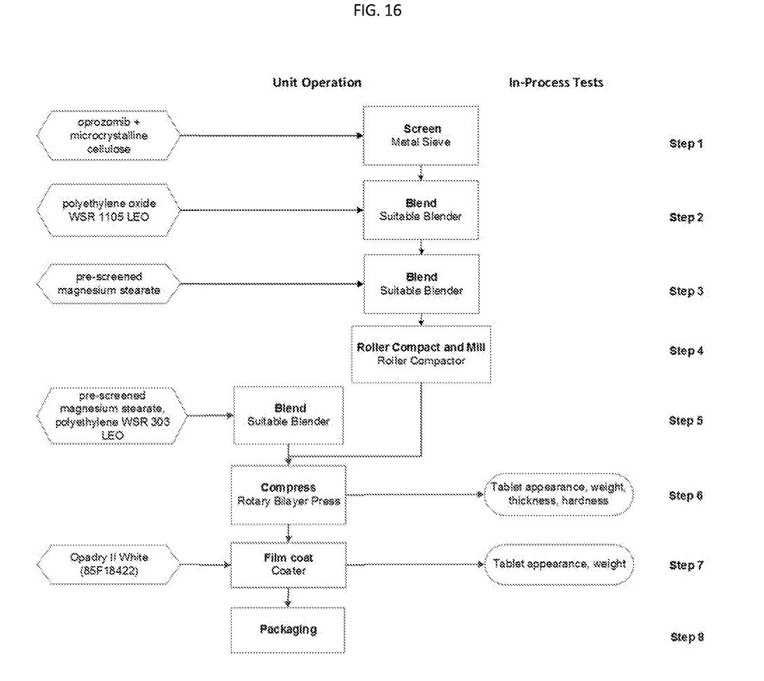

The flow diagram of the manufacturing process of the drug product is shown in FIG. 16.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Accordingly, other embodiments are within the scope of the following claims.

In some embodiments, any one or more of the features described throughout the specification may be combined with any one or more of the features described throughout the specification.

All of the above-cited references and publications are hereby incorporated by reference.

What is claimed:

1. A gastro-retentive modified release oral drug dosage form for releasing a sparingly soluble active pharmaceutical agent into the stomach, duodenum and/or upper small intestine of a patient, the drug dosage form comprising:
a. a first layer, the first layer comprising a first swellable polymer;
b. a second layer, the second layer comprising a second swellable polymer;
c. the first swellable polymer swells via imbibition of water from gastric fluid to promote gastric retention in the stomach of the patient;
d. the second swellable polymer swells via imbibition of water from gastric fluid to promote gastric retention in the stomach of the patient;
e. each first and second swellable polymer gradually erodes over a time period of hours, the erosion commencing upon contact with the gastric fluid, wherein the erosion releases the sparingly soluble pharmaceutical agent to the stomach, duodenum and/or upper small intestine of the patient as a result of the erosion at a rate corresponding to the time period;
f. wherein the first and second swellable polymers each comprises polyethylene oxide;
g. a sparingly soluble active pharmaceutical agent dispersed within at least one of the first and second layers; and
h. wherein the sparingly soluble active pharmaceutical agent is oprozomib or a pharmaceutically acceptable salt thereof.

2. The dosage form in accordance with claim 1 wherein the first swellable polymer decreases the release rate of the sparingly soluble active pharmaceutical agent.

3. The dosage form according to claim 1 wherein the first layer is granulated.

4. The dosage form according to claim 1 wherein the oprozomib is dispersed within the first layer.

5. The dosage form according to claim 1 wherein the oprozomib is dispersed within the first swellable polymer.

6. The dosage form according to claim 1 wherein the first and second swellable polymers exhibit different swelling and erosion rates.

7. The dosage form according to claim 1 wherein the second swellable polymer swells at a faster rate and erodes at a slower rate than the first polymer.

8. The dosage form according to claim 1, wherein the dosage form is in a form suitable for oral administration.

9. The dosage form according to claim 1, wherein the dosage form is a solid tablet.

10. The dosage form according to claim 1, wherein the dosage form is a bilayer tablet.

11. The dosage form according to claim 1, wherein the patient is in a fed mode.

12. The dosage form according to claim 1, wherein the dosage form comprises from about 2 weight percent to about 50 weight percent of oprozomib, or a pharmaceutically acceptable salt thereof.

13. The dosage form according to claim 1, wherein the dosage form comprises from about 3 weight percent to about 30 weight percent of oprozomib, or a pharmaceutically acceptable salt thereof.

14. The dosage form according to claim 1, wherein the dosage form comprises from about 4 weight percent to about 10 weight percent of oprozomib, or a pharmaceutically acceptable salt thereof.

15. The dosage form according to claim 1, wherein the dosage form comprises about 4.17 weight percent of oprozomib, or a pharmaceutically acceptable salt thereof.

16. The dosage form according to claim 1, wherein the dosage form comprises about 25 milligrams of oprozomib, or a pharmaceutically acceptable salt thereof.

17. The dosage form according to claim 1, wherein the dosage form comprises from about 4.17 weight percent to about 5 weight percent of oprozomib, or a pharmaceutically acceptable salt thereof; and about 25 milligrams of oprozomib, or a pharmaceutically acceptable salt thereof.

18. The dosage form according to claim 1, wherein the dosage form comprises about 4.17 weight percent of oprozomib, or a pharmaceutically acceptable salt thereof; and about 25 milligrams of oprozomib, or a pharmaceutically acceptable salt thereof.

19. The dosage form according to claim 1, wherein the dosage form comprises about 16.67 weight percent of oprozomib, or a pharmaceutically acceptable salt thereof.

20. The dosage form according to claim 1, wherein the dosage form comprises from about 20 weight percent to about 25 weight percent of oprozomib, or a pharmaceutically acceptable salt thereof.

21. The dosage form according to claim 1, wherein the dosage form comprises about 20 weight percent of oprozomib, or a pharmaceutically acceptable salt thereof.

22. The dosage form according to claim 1, wherein the dosage form comprises about 150 milligrams of oprozomib, or a pharmaceutically acceptable salt thereof.

23. The dosage form according to claim 1, wherein the dosage form comprises from about 20 weight percent to about 25 weight percent of oprozomib, or a pharmaceutically acceptable salt thereof; and about 150 milligrams of oprozomib, or a pharmaceutically acceptable salt thereof.

24. The dosage form according to claim 1, wherein the dosage form comprises about 25 weight percent of oprozomib, or a pharmaceutically acceptable salt thereof; and about 200 milligrams of oprozomib, or a pharmaceutically acceptable salt thereof.

26. The dosage form according to claim 1, wherein the dosage form comprises about 33.33 weight percent of oprozomib, or a pharmaceutically acceptable salt thereof.

27. The dosage form according to claim 1, wherein the dosage form comprises about 100 milligrams of oprozomib, or a pharmaceutically acceptable salt thereof.

28. The dosage form according to claim 1, wherein the dosage form comprises about 300 milligrams of oprozomib, or a pharmaceutically acceptable salt thereof.

29. The dosage form according to claim 1, wherein the dosage form comprises about 600 milligrams of oprozomib, or a pharmaceutically acceptable salt thereof.

30. The dosage form according to claim 1, wherein the dosage form comprises about 50 weight percent of oprozomib, or a pharmaceutically acceptable salt thereof; and about 300 milligrams of oprozomib, or a pharmaceutically acceptable salt thereof.

31. The dosage form according to claim 1, wherein the dosage form comprises about 50 weight percent of oprozomib, or a pharmaceutically acceptable salt thereof; and about 600 milligrams of oprozomib, or a pharmaceutically acceptable salt thereof.

32. The dosage form according to claim 1, wherein the dosage form comprises from about 15 weight percent to about 20 weight percent of oprozomib, or a pharmaceutically acceptable salt thereof; and about 100 milligrams of oprozomib, or a pharmaceutically acceptable salt thereof.

33. The dosage form according to claim 1, wherein the dosage form comprises about 16.67 weight percent of oprozomib, or a pharmaceutically acceptable salt thereof; and about 100 milligrams of oprozomib, or a pharmaceutically acceptable salt thereof.

34. The dosage form according to claim 1, wherein the dosage form comprises about 20 weight percent of oprozomib, or a pharmaceutically acceptable salt thereof; and about 150 milligrams of oprozomib, or a pharmaceutically acceptable salt thereof.

35. The dosage form according to claim 1, wherein the dosage form comprises about 24.24 weight percent of oprozomib, or a pharmaceutically acceptable salt thereof; and about 200 milligrams of oprozomib, or a pharmaceutically acceptable salt thereof.

36. The dosage form according to claim 1, wherein the oprozomib, or pharmaceutically acceptable salt thereof, is a crystalline solid.

37. The dosage form according to claim 1, wherein the oprozomib, or pharmaceutically acceptable salt thereof, is an amorphous solid.

38. The dosage form according to claim 1, wherein the dosage form further comprises one or more fillers.

39. The dosage form of claim 38, wherein the one or more fillers is microcrystalline cellulose.

40. The dosage form according to claim 1, wherein the first swellable polymer comprises from about 2 weight percent to about 80 weight percent of the total weight of the dosage form.

41. The dosage form according to claim 1, wherein the first swellable polymer comprises from about 15 weight percent to about 75 weight percent of the total weight of the dosage form.

42. The dosage form according to claim 1, wherein the first swellable polymer comprises from about 5 weight percent to about 20 weight percent of the total weight of the dosage form.

43. The dosage form according to claim 1, wherein the polyethylene oxide is PolyOx WSR 1105 LEO® polymer.

44. The dosage form according to claim 1, wherein the second swellable polymer comprises from about 2 weight percent to about 80 weight percent of the total weight of the dosage form.

45. The dosage form according to claim 1, wherein the second swellable polymer comprises from about 10 weight percent to about 65 weight percent of the total weight of the dosage form.

46. The dosage form according to claim 45, wherein the second swellable polymer comprises from about 16 weight percent to about 30 weight percent of the total weight of the dosage form.

47. The dosage form according to claim 1, wherein the second swellable polymer has a greater molecular weight than the first swellable polymer.

48. The dosage form according to claim 1, wherein the dosage form further comprises one or more lubricants.

49. The dosage form of claim 48, wherein the one or more lubricants is magnesium stearate.

50. The dosage form according to claim 1, wherein the dosage form further comprises one or more coatings.

51. The dosage form according to claim 50, wherein the one or more coatings is Opadry II White® (85F18422) coating.

52. The dosage form according to claim 1, wherein the tablet has a thickness of from about 2.5 millimeters to about 8 millimeters.

53. The dosage form according to claim 52, wherein the tablet has a thickness of from about 2.5 millimeters to about 4 millimeters.

54. The dosage form according to claim 1, wherein the tablet has a hardness of from about 1.00 kp to about 50.00 kp.

55. The dosage form according to claim 1, wherein the dosage form comprises:
a. a first layer comprising, by total weight percent (wt %) of the dosage form,
i) about 4% oprozomib;
ii) about 62% microcrystalline cellulose;
iii) about 12% PolyOx® WSR 1105 LEO® polymer, and
iv) about 0.50% magnesium stearate;
b. a second layer comprising, by total weight percent (wt %) of the dosage form,
i) about 21% PolyOx® WSR 303 LEO® polymer; and
ii) about 0.50% magnesium stearate; and
c. a film coating.

56. The dosage form according to claim 1, wherein the dosage form comprises;
a. a first layer, comprising, by total weight percent (wt %) of the dosage form:
i) about 17% oprozomib;
ii) about 49% microcrystalline cellulose;
iii) about 8% PolyOx® WSR 1105 LEO® polymer; and
iv) about 0.50% magnesium stearate;
b. a second layer, comprising, by total weight percent (wt %) of the dosage form:
i) about 25% PolyOx® WSR 303 LOE® polymer; and
ii) about 0.50% magnesium stearate; and
c. a film coating.

57. The dosage form according to claim 1, wherein the dosage form comprises;
a. a first layer, comprising, by total weight percent (wt %) of the dosage form:
i) about 20% oprozomib;
ii) about 49% microcrystalline cellulose;
iii) about 11% PolyOx® WSR 1105 LEO® polymer; and
iv) about 0.8% magnesium stearate;
b. a second layer, comprising, by total weight percent (wt %) of the dosage form:
i) about 20% PolyOx® WSR 303 LOE® polymer; and
ii) about 0.2% magnesium stearate; and
c. a film coating.

58. The dosage form according to claim 1, wherein the dosage form comprises;
a. a first layer, comprising, by total weight percent (wt %) of the dosage form:
i) about 24% oprozomib;
ii) about 48% microcrystalline cellulose;
iii) about 9% PolyOx® WSR 1105 LEO® polymer; and
iv) about 0.8% magnesium stearate;
b. a second layer, comprising, by total weight percent (wt %) of the dosage form:
i) about 18% PolyOx® WSR 303 LOE® polymer; and
ii) about 0.2% magnesium stearate; and
c. a film coating.

59. The dosage form according to claim 1, wherein the dosage form provides oprozomib with time to peak plasma concentrations of from about 1 hour to about 8 hours.

60. The dosage form according to claim 1, wherein the dosage form provides oprozomib with time to peak plasma concentrations of from about 4 hours to about 8 hours.

61. The dosage form according to claim 1, wherein the dosage form provides oprozomib with time to peak plasma concentrations of from about 4 hours to about 6 hours.

62. The dosage form according to claim 1, wherein the dosage form provides oprozomib with time to peak plasma concentrations of about 8 hours.

63. The dosage form according to claim 1, wherein the dosage form provides oprozomib from about less than or equal to 40% of a preferred dose at approximately 1 hour.

64. The dosage form according to claim 1, wherein the dosage form provides oprozomib from about 40% to about 75% of the preferred dose at approximately 4 hours.

65. The dosage form according to claim 1, wherein the dosage form provides oprozomib from about greater than or equal to 75% of the preferred dose at approximately 8 hours.

66. The dosage form according to claim 1, wherein a single dose of the dosage form comprising about 60 mg of oprozomib to a dog produces peak plasma concentration (Cₘₐₓ) of oprozomib of 3.81 ng/mL (having a standard deviation of 2.28).

67. The dosage form of claim 66, wherein a single dose of the dosage form to a dog produces an area under the concentration time curve to the last time point (AUC) of oprozomib of 15.6 ng*hr/mL (having a standard deviation of 17.4).

68. The dosage form according to claim 1, wherein the dosage form is stable upon actual or simulated storage at 25° C./60% relative humidity for at least 1 month.

69. The dosage form according to claim 1, wherein equal to or more than about 75% of oprozomib, or a pharmaceutically acceptable salt thereof, is released within about 8 hours as determined by UV under the following dissolution conditions:

| | |
|---|---|
| Dissolution medium | About 0.01N Hydrochloric acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all rows |
| Residence Time | 2 hrs in first row |
| | 3 hrs in second row |
| | 3 hrs in third row |
| Sampling | Manual or automatic |
| Sampling Time | About 1, 2, 3, 4, 5, 7, 8 hours |
| Sampling Volume | About 5 mL |
| Screen size | 20 mesh |
| Filter | 5 um Titan 2 syringe filters (used new |
| | filters for each sampling time point) |
| UV Detection | 258 nm |
| Cell path length | 2 mm for 100 mg |
| (for standard UV | 10 mm for 25 mg |
| spectrophotometer) | |

70. The dosage form according to claim 1, wherein equal to or more than about 75% of oprozomib, or a pharmaceutically acceptable salt thereof, is released within about 8 hours as determined by UV under the following dissolution conditions:

| | |
|---|---|
| Dissolution medium | About 0.01N Hydrochloric |
| | acid (HCl) |
| Media volume | About 250 mL for all rows |
| Number of rows | 3 |
| Temperature | 37 ± 0.5° C. |
| Apparatus | USP No. 3 Dissolution |
| Agitation Speed | 30 Dips/minute (DPM)for all |
| | rows |
| Residence Time | 2 hrs in first row |
| | 2 hrs in second row |
| | 2 hrs in third row |
| | 2 hrs in fourth row |
| Sampling | Manual |
| Sampling Time | About 1, 2, 3, 4, 5, 7, 8 hours |
| Sampling Volume | About 5 mL |
| Screen size | 20 mesh |
| Filter | 5 um Titan 2 syringe filters |
| | (used new filters for each sampling |
| | time point) |
| UV Detection | 258 nm |
| Cell path length (for | 1 mm for 150 mg and 200 mg |
| standard UV | 2 mm for 100 mg |
| spectrophotometer Cary ® UV | 10 mm for 25 mg |
| 50, Varian, Inc.)) | |

71. The dosage form of claim 70, wherein from about less than or equal to 40% of a preferred dose of oprozomib is released at approximately 1 hour.

72. The dosage form of claim 69, wherein from about 40% to about 75% of the preferred dose of oprozomib is released at approximately 4 hours.

73. The dosage form of claim 70, wherein from about 40% to about 75% of the preferred dose of oprozomib is released at approximately 4 hours.

74. The dosage form according to claim 1, wherein the dosage form provides a reduced incidence or severity of one or more gastrointestinal (GI) side effects.

75. The dosage form of claim 74, wherein of one or more gastrointestinal (GI) side effects include one or more of nausea, vomiting or emesis, increased salivation and diarrhea.

76. The dosage form of claim 75, wherein the side effects include vomiting or emesis.

77. The dosage form of claim 75, wherein the side effects include increased salivation.

78. The dosage form according to claim 1, wherein the dosage form is prepared by dry granulation.

79. A method for treating a disease or condition selected from the group consisting of cancer, autoimmune disease, graft or transplant-related condition, neurodegenerative disease, fibrotic-associated condition, ischemic-related conditions, infection (viral, parasitic or prokaryotic) and diseases associated with bone loss, the method comprising administering a dosage form as claimed in claim 1.

80. The method of claim 79, wherein the disease or condition is cancer.

81. The method of claim 80, wherein the cancer is selected from multiple myeloma, Waldenström's macroglobulinemia, chronic lymphocytic leukemia, and myelodysplastic syndromes.
